(19) United States
(12) Patent Application Publication
(10) Pub. No.: US 2023/0095372 A1
(43) Pub. Date: Mar. 30, 2023

(54) MULTICYCLIC CARBOCATION AND CARBORADICAL COMPOUNDS AND METHODS OF USE

(71) Applicant: Arizona Board of Regents on Behalf of the University of Arizona, Tucson, AZ (US)

(72) Inventor: Thomas L. Gianetti, Tucson, AZ (US)

(21) Appl. No.: 17/793,204

(22) PCT Filed: Jan. 31, 2021
(86) PCT No.: PCT/US2021/015961

§ 371 Date: Jul. 15, 2022

Related U.S. Application Data
(60) Provisional application No. 62/968,880, filed on Jan. 31, 2020.
(60) Provisional application No. 63/025,915, filed on May 15, 2020.

(51) Int. Cl. C07F 15/06; B01J 35/00; C07D 471/06; C07F 5/02; C07F 15/04; B01J 31/18

(52) U.S. Cl. CPC C07F 15/065; B01J 35/004; C07D 471/06; C07F 5/022; C07F 15/045; B01J 31/183

(57) ABSTRACT

The present invention provides a compound comprising a moiety of the formula: (I) where said moiety of formula I is a radical, a cation, or a radical dication; Y¹, Y², Y³, R^{1a}, R^{1b}, R^{1c}, R^{1d}, R^{2a}, R^{2b}, R^{2c}, R^{2d}, R^{3a}, R^{3b}, R^{3c}, and R^{3d} are as defined herein. Compounds containing a moiety of Formula I are useful in a wide variety of applications including, but not limited to, as photocatalysts, use in OLEDs, in electronic components, etc. As an organic-based photocatalysts, compounds containing a moiety of Formula I are activated by a relatively low energy electromagnetic wavelength, e.g., wavelength of 600 nm or greater. Furthermore, compounds of the invention can be used as both photoreduction catalysts and photooxidation catalysts.

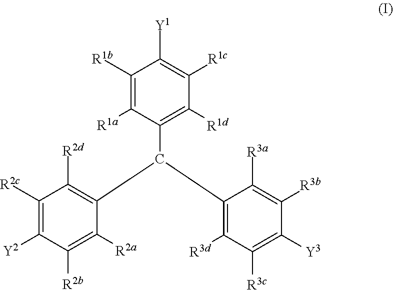

### CROSS-REFERENCE TO RELATED APPLICATIONS

This application claims the priority benefit of U.S. Provisional Application Nos. 62/968,880, filed Jan. 31, 2020, and 63/025,915, filed May 15, 2020 which are incorporated herein by reference in their entirety.

### FIELD OF THE INVENTION

The present invention relates to multicyclic carbocation and carboradical compounds and methods of use. In particular, the present invention relates to a compound comprising a moiety of the formula:

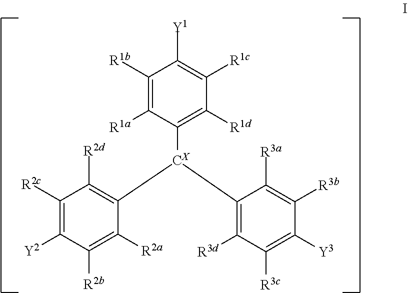

where said moiety of Formula I is a radical, a cation, or a radical dication; “C^{X}” denotes central carbon; Y¹, Y², Y³, R^{1a}, R^{1b}, R^{1c}, R^{1d}, R^{2a}, R^{2b}, R^{2c}, R^{2a}, R^{3a}, R^{3b}, R^{3c}, and R^{3d} are as defined herein. Compounds containing a moiety of Formula I are useful in a wide variety of applications including, but not limited to, as photocatalysts. In particular, compounds containing a moiety of Formula I are activated by a relatively low energy electromagnetic wavelength, e.g., wavelength of 600 nm or greater.

### BACKGROUND OF THE INVENTION

Molecular catalysis plays an important role in a wide variety of applications including, but not limited to, chemical reactions, photosynthesis, electron transfer (e.g., in photovoltaic cells), etc. Catalyst may be classified as either homogeneous or heterogeneous depending on whether the catalyst is dispersed in the same phase (usually gaseous or liquid) as the reactant's molecules or solvent. A heterogeneous catalyst is typically used as a solid catalyst that are often adsorbed onto the surface of a solid material.

Molecular metal-based photocatalysts (“PCs”) have many advantages as well as disadvantages. Some of the advantages of metal-based PCs include long half-life time, wide range of oxidation and reduction potential, ability to provide both oxidative quenching and reductive quenching. Unfortunately, many metal-based PCs are expensive, toxic, environmentally unfriendly, and require high energy electromagnetic radiation, typically in the blue-light region or higher energy (e.g., hv of less than about 460 nm).

To overcome some of the disadvantages of metal-based PCs, organic-based PCs have recently been developed. These organic-based PCs have advantages of being less expensive and more environmentally friendly relative to metal-based PCs. Unfortunately, however, conventional organic-based PCs suffer from short-half life. Moreover, unlike metal-based PCs, organic-based PCs cannot be used to provide both oxidative quenching and reductive quenching. In addition, majority of organic PCs also require blue light or higher energy electromagnetic radiation for activation. While few organic PCs can use lower energy electromagnetic radiation for activation, such organic PCs are relatively difficult to synthesize and/or tune electromagnetic radiation wavelength for activation.

Therefore, there is a continuing need for organic-based PCs that can be activated with a relative low energy electromagnetic radiation, have a relatively longer half-life, environmentally friendly, and/or readily synthesized.

### SUMMARY OF THE INVENTION

The present invention provides a radical, a cation, and a radical dication moieties that are useful in a wide variety of applications including, but not limited to, as photocatalysts and components in various electrochemical processes. One aspect of the invention provides a compound comprising a moiety of the formula I.

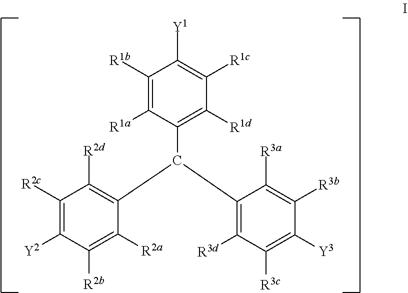

where said moiety is a radical, a cation, or a radical dication; each of R^{1a}, R^{1b}, R^{1c}, R^{1d}, R^{2a}, R^{2b}, R^{2c}, R^{2a}, R^{3a}, R^{3b}, R^{3c}, and R^{3d} is independently H, halide, haloalkyl (e.g., CF₃), —NR^{a}R^{b}(each of R^{a} and R^{b} is independently H or C₁-C₄ alkyl), C₁-C₁₂ alkyl, C₁-C₄ alkoxy, —NO₂, —CN, —CO₂R (wherein R is H or C₁-C₄ alkyl), or Ar¹; or R^{2a} and R^{3d} together form —X¹—; or R^{1a} and R^{2d} together form —X²—; or R^{1d} and R^{3a} together form —X³—; each of X¹, X² and X³ is independently O, NR^{4a}, PR^{4a}, CR^{4a}R^{4b}, or SiR^{4a}R^{4b}; or R^{1a} and R^{1b} together with atoms to which they are attached to form an optionally substituted aromatic ring (e.g., optionally substituted phenyl); or R^{2c} and R^{2d} together with atoms to which they are attached to form an optionally substituted aromatic ring (e.g., optionally substituted phenyl); each of Y¹, Y², and Y³ is independently H, OR^{5a}, CR^{5a}R^{5b}R^{5b} NR^{5a}R^{5b} PR^{5a}R^{5b}, NO₂, CN, haloalkyl (e.g., CF₃), CO₂R, N₃, or Ar¹; each of R^{4a}, R^{4b}, R^{5a}, and R^{5b} is independently H, halide, haloalkyl (e.g., CF₃), C₁-C₁₂ alkyl, C₁-C₄ alkoxy, —NR^{a}R^{b} (provided at least one of R^{a} and R^{b} is C₁-C₄ alkyl), Ar¹, or a moiety of the formula -L-Z, where L is a linker; and Z is a heterocyclic species of Formula I (i.e., dimer, trimer, or other oligomers of formula I), a coordinating group able to bind or complex to a metal ion, or a water-soluble group such as phosphate, phosphite, sulfate or sulfite; and Ar¹ is optionally substituted aryl (e.g., optionally substituted phenyl) having from 0 to 5 substituents, each of which is independently selected from the group consisting of X¹, haloalkyl (e.g., CF₃), NR^{a}R^{b}, C₁-C₄ alkyl, and C₁-C₄ alkoxy, heteroaryl, fused aryl or Ar¹, provided when said Formula I is a cation then (i) when R^{2a} and R^{3d} together form —X¹—; R^{1a} and R^{2d} together form —X²—; and R^{1d} and R^{3a} together form —X³—, then no more than one of X¹, X², and X³ is 0; (ii) when R^{2a} and R^{3d} together form —X¹—, and R^{1a} and R^{2d} together does not form —X²—; and R^{1d} and R^{3a} together does not form —X³—, then at least one of R^{1a}, R^{1b}, R^{1c}, R^{1d}, or Y¹ is not H, halogen, or C₁-C₁₂ alkyl; (iii) at least one of (a) R^{2a} and R^{3d} together form —X¹—; (b) R^{1a} and R^{2d} together form —X²—; or (c) R^{1d} and R^{3a} together form —X³—; and (iv) when R^{2a} and R^{3d} together form —X¹—; R^{1a} and R^{2d} together form —X²—; and R^{1d} and R^{3a} together form —X³—, then none of X¹, X², and X³ is NR^{4a}.

In some embodiments, Y¹ is NR^{5a}R^{5b}, PR^{5a}R^{5b}, haloalkyl (e.g., CF₃), N₃, or Ar¹. Yet in other embodiments, said moiety of Formula I is selected from the group consisting of:

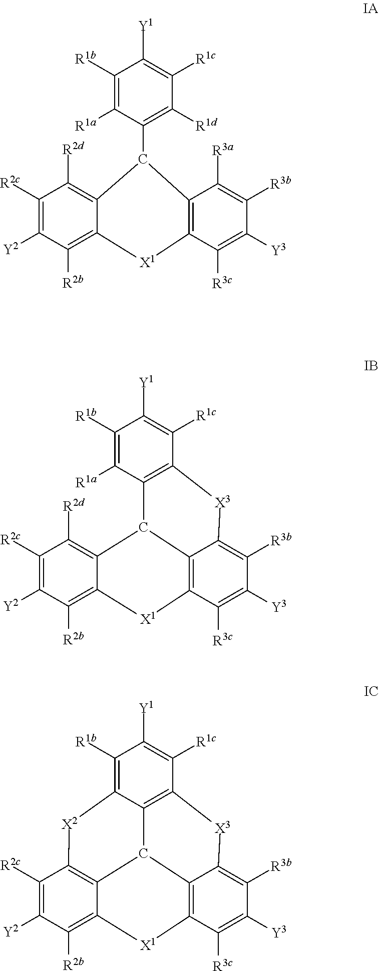

where Y¹, Y², Y³, X¹, X², X³, R^{1a}, R^{1b}, R^{1c}, R^{1d}, R^{2b}, R^{2c}, R^{2a}, R^{3a}, R^{3b}, and R^{3c} are those defined herein. Still in some embodiments, said moiety of Formula IB or IC is a radical or a radical dication. In further embodiments, X¹ is NR^{4a}.

In other embodiments, R^{4a} is a moiety of the formula L-Z. Yet in some embodiments, Z is a coordinating group selected from the group consisting of bipyridinyl, pyridinyl, —PR₂, —OPR₂, —NHC, —NR₂, diimine, imine, —OH, —OR, —SR, —SH, diphosphines, —RNC, —CO₂H, and carboiimine, where each R is independently C₁-C₁₂ alkyl or Aryl. Still in other embodiments, L is C₁-C₁₀ alkylene, alkynylene, alkenylene, arylene, or heteroarylene.

Still in another embodiment, each of R^{1c}, R^{2c}, and R^{3c} is independently C₁-C₄ alkoxy. Yet in another embodiment, Y, R^{1a}, R^{1b}, R^{2a}, R^{2b}, R^{3a}, and R^{3b} are H.

Another aspect of the invention provides a radical or a diradical cation moiety of the formula:

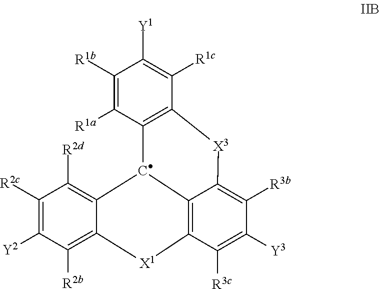

where each of R^{1a}, R^{1b}, R^{1c}, R^{2b}, R^{2c}, R^{2a}, R^{3b}, and R^{3c} is independently H, halide, haloalkyl (e.g., CF₃), —NR^{a}R^{b}, C₁-C₁₂ alkyl, C₁-C₄ alkoxy, —NO₂, —CN, —CO₂R (wherein R is H or C₁-C₄ alkyl), or Ar¹; or R^{1a} and R^{2d} together form —X²—; each of R^{a} and R^{b} is independently H or C₁-C₄ alkyl; each of X¹, X² and X³ is independently O, NR^{4a}, PR^{4a}, CR^{4a}R^{4b}, or SiR^{4a}R^{4b}; or R^{1a} and R^{1b} together with atoms to which they are attached to form an optionally substituted aromatic ring (e.g., optionally substituted phenyl); or R^{2c} and R^{2d} together with atoms to which they are attached to form an optionally substituted aromatic ring (e.g., optionally substituted phenyl); each of Y¹, Y², and Y³ is independently H, OR^{5a}, NR^{5a}R^{5b} PR^{5a}R^{5b}, NO₂, CN, CF₃, CO₂R, N₃, or Ar¹; each of R^{4a}, R^{4b}, R^{5a}, and R^{5b} is independently H, halide, haloalkyl (e.g., CF₃), C₁-C₁₂ alkyl, C₁-C₄ alkoxy, —NR^{a}R^{b} (provided at least one of R^{a} and R^{b} is C₁-C₄ alkyl and provided R^{4a} is not attached to N), Ar¹, or a moiety of the formula -L-Z, where L is a linker; and Z is a coordinating group able to bind or complex to a metal ion; and Ar¹ is optionally substituted phenyl having from 0 to 5 substituents, each of which is independently selected from the group consisting of X¹, haloalkyl (e.g., CF₃), NR^{a}R^{b}, C₁-C₄ alkyl, and C₁-C₄ alkoxy.

In further embodiments, X¹ and X³ are NR^{4a}, wherein R^{4a} is as defined herein. In some instances, each R^{4a} is independently C₁-C₁₂ alkyl or a moiety of the formula -L-Z. Yet in other instances, Z is selected from the group consisting of a heteroaryl, heterocyclyl, and a heteroatom functional group (e.g., NH2, etc.). In still other instances, Y¹, Y², and Y³ are NR^{5a}R^{5b}, where each of R^{5a} and R^{5b} is independently H, haloalkyl (e.g., CF₃) or C₁-C₁₂ alkyl, provided at least one of R⁵ and R^{5b} is not H. In other embodiments, R^{1a} and R^{2d} are C₁-C₄ alkoxy.

Yet another aspect of the invention provides a photocatalytic compound that for capable of catalyzing an oxidative reaction and a reductive reaction, said photocatalytic compound comprising a compound comprising a moiety of Formula I. In some embodiments, said photocatalytic compound is activated by an electromagnetic radiation having wavelength of 390 nm or greater.

Still another aspect of the invention provides a method for producing an oxidative product from a reaction substrate, said method comprising: contacting said reaction substrate with an oxidizing reagent, and a photocatalytic compound comprising a moiety of Formula I to produce a reaction mixture; and irradiating said reaction mixture with an electromagnetic radiation having a wavelength of 390 nm or greater under conditions sufficient to produce said oxidative product from said reaction substrate. In some embodiments, said method comprises an aerobic oxidative hydroxylation or aerobic oxygenation. In some instances, said aerobic oxygenation comprises oxygenation of benzylic carbon.

Another aspect of the invention provides a method for producing a reductive coupling product from a reaction substrate, said method comprising: contacting said reaction substrate with an oxidizing reagent, and a photocatalytic compound comprising a moiety of Formula I to produce a reaction mixture; and irradiating said reaction mixture with an electromagnetic radiation having a wavelength of 390 nm or greater under conditions sufficient to produce said reductive coupling product from said reaction substrate. In some embodiments, said method comprises dual catalysis arylation reaction of an sp²-carbon atom or atom transfer radical reaction (ATRA).

Still another aspect of the invention provides solid substrate having a surface, wherein said surface comprises a compound comprising a moiety of the formula:

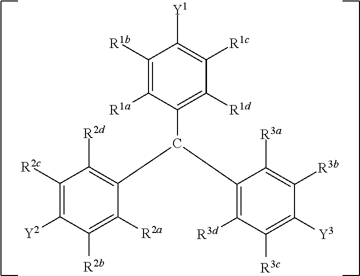

where said moiety is a radical, a cation, or a radical dication; each of R^{1a}, R^{1b}, R^{1c}, R^{1d}, R^{2a}, R^{2b}, R^{2c}, R^{2a}, R^{3a}, R^{3b}, R^{3c}, and R^{3d} is independently H, halide, haloalkyl (e.g., CF₃), —NR^{a}R^{b}, C₁-C₁₂ alkyl, C₁-C₄ alkoxy, —NO₂, —CN, —CO₂R (wherein R is H or C₁-C₄ alkyl), or Ar¹; or R^{2a} and R^{3d} together form —X¹—; or R^{1a} and R^{2d} together form —X²—; or R^{1d} and R^{3a} together form —X³—; each of R^{a} and R^{b} is independently H or C₁-C₄ alkyl; each of X¹, X² and X³ is independently O, NR^{4a}, PR^{4a}, CR^{4a}R^{4b}, or SiR^{4a}R^{4b}; or R^{1a} and R^{1b} together with atoms to which they are attached to form an optionally substituted aromatic ring (e.g., optionally substituted phenyl); or R^{2c} and R^{2d} together with atoms to which they are attached to form an optionally substituted aromatic ring (e.g., optionally substituted phenyl); each of Y¹, Y², and Y³ is independently H, OR^{5a}, CR^{5a}R^{5b}R^{5b}, NR^{5a}R^{5b} PR^{5a}R^{5b}, NO₂, CN, CF₃, CO₂R, N₃, or Ar¹; each of R^{4a}, R^{4b}, R^{5a}, and R^{5b} is independently H, halide, haloalkyl (e.g., CF₃), C₁-C₁₂ alkyl, C₁-C₄ alkoxy, —NR^{a}R^{b} (provided at least one of R^{a} and R^{b} is C₁-C₄ alkyl), Ar¹, or a moiety of the formula -L-Z, wherein L is a linker; and Z is a heterocyclic species of formula I (i.e., dimer, trimer, or other oligomers of formula I), a coordinating group able to bind or complex to a metal ion, or a water-soluble group such as phosphate, phosphite, sulfate or sulfite; and Ar is optionally substituted aryl (e.g., phenyl) having from 0 to 5 substituents, each of which is independently selected from the group consisting of X¹, haloalkyl (e.g., CF₃), NR^{a}R^{b}, C₁-C₄ alkyl, and C₁-C₄ alkoxy, heteroaryl, fused aryl or Ar¹.

Yet in other embodiments, said moiety of Formula I is selected from the group consisting of:

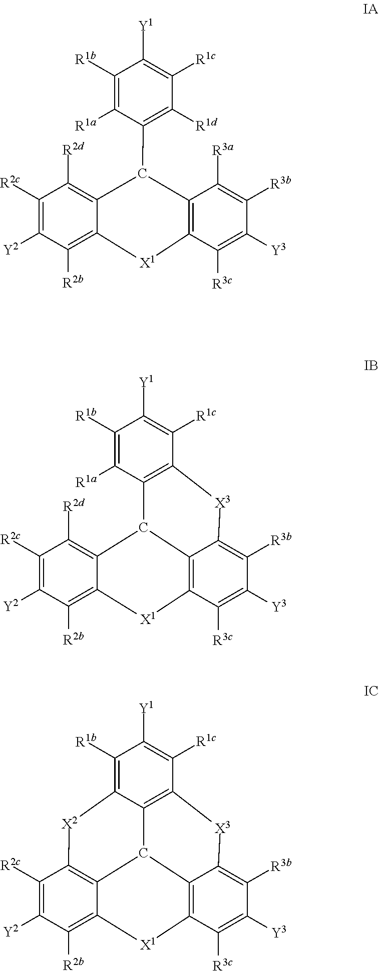

wherein Y¹, Y², Y³, X¹, X², X³, R^{1a}, R^{1b}, R^{1c}, R^{1d}, R^{2b}, R^{2c}, R^{2d}, R^{3a}, R^{3b}, and R^{3c} are as defined herein. In some instances, said moiety of Formula IB or IC is a radical or a radical dication.

Still in other embodiments, when said moiety of Formula I is a cation then (i) when R^{2a} and R^{3d} together form —X¹—; R^{1a} and R^{2d} together form —X²—; and R^{1d} and R^{3a} together form —X³—, then no more than one of X¹, X², and X³ is O; (ii) when R^{2a} and R^{3d}together form —X¹—, and R^{1a} and R^{2d} together does not form —X²—; and R^{1d} and R^{3a} together does not form —X³—, then Y¹ is NR^{5a}R^{5b}, PR^{5a}R^{5b}, haloalkyl (e.g., CF₃), N₃, or Ar¹; (iii) at least one of (a) R^{2a} and R^{3d} together form —X¹—; (b) R^{1a} and R^{2d} together form —X²—; or (c) R^{1d} and R^{3a} together form —X³—; and (iv) when R^{2a} and R^{3d} together form —X¹—; R^{1a} and R^{2d} together form —X²—; and R^{1d} and R^{3a} together form —X³—, then none of X¹, X², and X³ is NR^{4a}.

Yet another aspect of the invention provides a method for conducting a photocatalytic reaction, said method comprising contacting two or more reagents in the presence of a photocatalyst compound under conditions sufficient to produce a photooxidative product or a photoreductive product, wherein said photocatalyst compound comprises a moiety of Formula I, where said moiety of formula I is a radical, a cation, or a radical dication; each of R^{1a}, R^{1b}, R^{1c}, R^{1d}, R^{2a}, R^{2b}, R^{2c}, R^{2a}, R^{3a}, R^{3b}, R^{3c}, and R^{3d} is independently H, halide, haloalkyl (e.g., CF₃), —NR^{a}R^{b}, C₁-C₁₂ alkyl, C₁-C₄ alkoxy, —NO₂, —CN, —CO₂R (wherein R is H or C₁-C₄ alkyl), or Ar¹; or R^{2a} and R^{3d} together form —X¹—; or R^{1a} and R^{2d} together form —X²—; or R^{1d} and R^{3a} together form —X³—; each of R^{a} and R^{b} is independently H or C₁-C₄ alkyl; each of X¹, X² and X³ is independently O, NR^{4a}, PR^{4a}, CR^{4a}R^{4b}, or SiR^{4a}R^{4b}; or R^{1a} and R^{1b} together with atoms to which they are attached to form an optionally substituted aromatic ring (e.g., optionally substituted phenyl); or R^{2c} and R^{2d} together with atoms to which they are attached to form an optionally substituted aromatic ring (e.g., optionally substituted phenyl); each of Y¹, Y², and Y³ is independently H, OR^{5a}, CR^{5a}R^{5b}R^{5b} NR^{5a}R^{5b}, PR^{5a}R^{5b}, NO₂, CN, CF₃, CO₂R, N₃, or Ar¹; each of R^{4a}, R^{4b}, R^{5a}, and R^{5b} is independently H, halide, haloalkyl (e.g., CF₃), C₁-C₁₂ alkyl, C₁-C₄ alkoxy, —NR^{a}R^{b} (provided at least one of R^{a} and R^{b} is C₁-C₄ alkyl), Ar¹, or a moiety of the formula -L-Z, where L is a linker; and Z is a heterocyclic species of formula I (i.e., dimer, trimer, or other oligomers of formula I), a coordinating group able to bind or complex to a metal ion, or a water-soluble group such as phosphate, phosphite, sulfate or sulfite; and Ar¹ is optionally substituted aryl (e.g., optionally substituted phenyl) having from 0 to 5 substituents, each of which is independently selected from the group consisting of X¹, haloalkyl (e.g., CF₃), NR^{a}R^{b}, C₁-C₄ alkyl, and C₁-C₄ alkoxy, heteroaryl, fused aryl or Ar¹, provided when said formula I is a cation when R^{2a} and R^{3d} together form —X¹—, and R^{1a} and R^{2d} together does not form —X²—; and R^{1d} and R^{3a} together does not form —X³—, then at least one of R^{1a}, R^{1b}, R^{1c}, R^{1d}, Y¹ is OR^{5a} NR^{5a}R^{5b} PR^{5a}R^{5b}, haloalkyl (e.g., CF₃), N₃, or Ar¹.

In some embodiments, said photocatalytic reaction is conducted using a red light. In some instances, said photocatalytic reaction is conducted using an electromagnetic radiation having a wavelength of 500 nm or greater.

In yet another aspect of the invention, a compound is provided that comprises a carbocation of the formula:

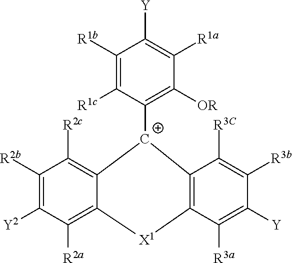

where R is C₁-C₁₂ alkyl or Aryl; X¹ is O, NR^{4a}, PR^{4a}, CR^{4a}R^{4b}, or SiR^{4a}R^{4b}; each of Y is independently H, OR^{5a} NR^{5a}R^{5b} PR^{5a}R^{5b}, NO₂, CN, haloalkyl, N₃ CO₂R, or Ar¹; each of R^{1a}, R^{1b}, R^{1c}, R^{2a}, R^{2b}, R^{2c}, R^{3a}, R^{3b}, and R^{3c}, is independently H, halide, CF₃, NH₂, C₁-C₁₂ alkyl, C₁-C₄ alkoxy, C₁-C₄ alkylamino, C₁-C₄ dialkyl amino, NO₂, CN, CO₂R, or Ar¹; each of each of R^{4a}, R^{4b}, R^{5a}, and R^{5b} is independently H, halide, CF₃, C₁-C₁₂ alkyl, C₁-C₄ alkoxy, C₁-C₄ alkylamino, C₁-C₄ dialkyl amino, or Ar¹; or R^{1c} and R^{2c} together form —X²—; or OR and R^{3c} together form —X³—; each of X² and X³ is independently O, NR^{4a}, PR^{4a}, CR^{4a}R^{4b}, or SiR^{4a}R^{4b}; or R^{1b} and R^{1c} together with atoms to which they are attached to form a phenyl; or R^{2b} and R^{2c} together with atoms to which they are attached to form a phenyl; and Ar¹ is optionally substituted phenyl having from 0 to 5 substituents, each of which is independently selected from the group consisting of X¹, CF₃, NH₂, C₁-C₄ alkyl, C₁-C₄ alkoxy, C₁-C₄ alkylamino, and C₁-C₄ dialkyl amino; or at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, wherein L is a linker; and Z is a coordinating group that is capable of coordinating to a metal complex. In some embodiments, at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, wherein L is a linker; and Z is a coordinating group that is capable of coordinating to a metal complex. Yet in another embodiment, the carbocation is of the formula:

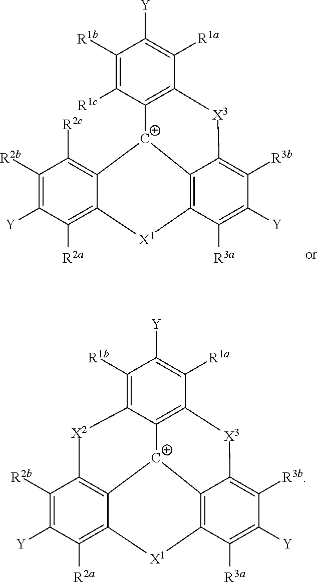

Still in some embodiments, X¹ is NR^{4a}. In another embodiment, R^{4a} is said moiety of the formula L-Z. In some instances, Z is said coordinating group selected from the group consisting of bipyridinyl, pyridinyl, —PR₂, —OPR₂, —NHC, —NR₂, diimine, imine, —OH, —OR, —SR, —SH, diphosphines, —RNC, —CO₂H, and carboiimine, and wherein each R is independently C₁-C₁₂ alkyl or Aryl. Still in other embodiments, Z is coordinated or complexed to a metal complex. In some instances, said metal ion is a transition metal ion. Still in other instances, said metal ion is a first row transition metal ion. Yet in other instances, said metal ion is selected from the group consisting of Sc(III), Ti(IV), V(III), V(V), Cr(III), Cr(IV), Mn(II), Co (II), Co(I), Ni (II), Fe (II), Fe(III), Cu(II), Cu(I), and Zn(I).

In further embodiments, L is C₁-C₁₀ alkylene, alkynylene, alkenylene, arylene, or heteroarylene. Yet in other embodiments, each of R^{1c}, R^{2c}, and R^{3c} is independently C₁-C₄ alkoxy. In one particular embodiment, Y, R^{1a}, R^{1b}, R^{2a}, R^{2b}, R^{3a}, and R^{3b} are H.

Yet another aspect of the invention provides a photocatalyst composition comprising a conjugated heterocyclic carbenium-metal complex of the formula:

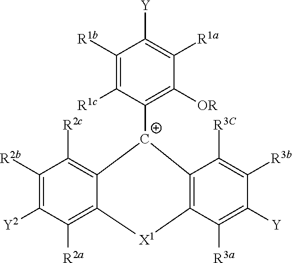

where R is C₁-C₁₂ alkyl or Aryl; X¹ is O, NR^{4a}, PR^{4a}, CR^{4a}R^{4b}, or SiR^{4a}R^{4b}; each of Y is independently H, OR^{1a} NR^{5a}R^{5b} PR^{5a}R^{5b}, NO₂, CN, haloalkyl, N₃ CO₂R, or Ar¹; each of R^{1a}, R^{1b}, R^{1c}, R^{2a}, R^{2b}, R^{2c}, R^{3a}, R^{3b}, and R^{3c}, is independently H, halide, CF₃, NH₂, C₁-C₁₂ alkyl, C₁-C₄ alkoxy, C₁-C₄ alkylamino, C₁-C₄ dialkyl amino, NO₂, CN, CO₂R, or Ar¹; each of each of R^{4a}, R^{4b}, R^{5a}, and R^{5b} is independently H, halide, CF₃, C₁-C₁₂ alkyl, C₁-C₄ alkoxy, C₁-C₄ alkylamino, C₁-C₄ dialkyl amino, or Ar¹; or R^{1c} and R^{2c} together form —X²—; or OR and R^{3c} together form —X³—; each of X² and X³ is independently O, NR^{4a}, PR^{4a}, CR^{4a}R^{4b}, or SiR^{4a}R^{4b}; or R^{1b} and R^{1c} together with atoms to which they are attached to form an optionally substituted phenyl; or R^{2b} and R^{2c} together with atoms to which they are attached to form an optionally substituted phenyl; and Ar is optionally substituted phenyl having from 0 to 5 substituents, each of which is independently selected from the group consisting of X¹, CF₃, NH₂, C₁-C₄ alkyl, C₁-C₄ alkoxy, C₁-C₄ alkylamino, and C₁-C₄ dialkyl amino; or at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, where L is a linker; and Z is a coordinating group that is coordinated to a metal complex. In some embodiments, said conjugated heterocyclic carbenium-metal complex is of the formula:

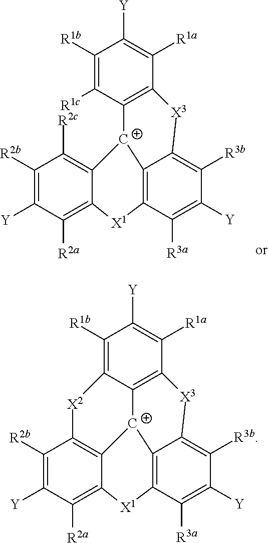

In other embodiments, X¹ is NR^{4a}, and R^{4a} is said moiety of the formula -L-Z.

Still in other embodiments, L has from 1 to about 10 chain of atoms. In some instances, L is C₁-C₁₀ alkylene or C₁-C₁₀ heteroalkylene.

Yet in other embodiments, said metal ion is a transition metal ion. In other embodiments, said metal ion is a first row transition metal ion. Still in other embodiments, said metal ion is selected from the group consisting of Co (II), Ni (II), and Fe (II).

Another aspect of the invention provides a solid substrate having a surface, wherein said surface comprises a compound comprising a carbocation of the formula:

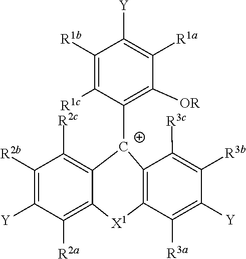

where R is C₁-C₁₂ alkyl or Aryl; X¹ is O, NR^{4a}, PR^{4a}, CR^{4a}R^{4b}, or SiR^{4a}R^{4b}; each of Y is independently H, OR^{5a}, NR^{5a}R^{5b} PR^{5a}R^{5b}, NO₂, CN, CO₂R, or Ar¹; each of R¹, R^{1b}, R^{1c}, R^{2a}, R^{2b}, R^{2c}, R^{3a}, R^{3b}, and R^{3c}, is independently H, halide, CF₃, NH₂, C₁-C₁₂ alkyl, C₁-C₄ alkoxy, C₁-C₄ alkylamino, C₁-C₄ dialkyl amino, NO₂, CN, CO₂R, or Ar¹; each of each of R^{4a}, R^{4b}, R^{5a}, and R^{5b} is independently H, halide, CF₃, C₁-C₁₂ alkyl, C₁-C₄ alkoxy, C₁-C₄ alkylamino, C₁-C₄ dialkyl amino, or Ar¹; or R^{1c} and R^{2c} together form —X²—; or OR and R^{3c} together form —X³—; each of X² and X³ is independently O, NR^{4a}, PR^{4a}, CR^{4a}R^{4b}, or SiR^{4a}R^{4b}; or R^{1b} and R^{1c} together with atoms to which they are attached to form an optionally substituted phenyl; or R^{2b} and R^{2c} together with atoms to which they are attached to form an optionally substituted phenyl; and Ar¹ is optionally substituted phenyl having from 0 to 5 substituents, each of which is independently selected from the group consisting of X¹, CF₃, NH₂, C₁-C₄ alkyl, C₁-C₄ alkoxy, C₁-C₄ alkylamino, and C₁-C₄ dialkyl amino; or at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, wherein L is a linker; and Z is a functional group attached to a surface of a solid substrate; or optionally at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L′-Z′, wherein L′ is a linker and Z′ is a metal complex.

In some embodiments, said carbocation is of the formula:

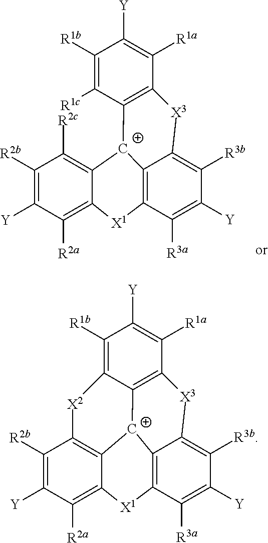

Yet in other embodiments, at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L′-Z′.

### BRIEF DESCRIPTION OF THE DRAWINGS

FIG. 1 shows a synthetic scheme for producing some of the cations and radicals of the invention.

FIG. 2 is cyclic voltammograms of compounds 2⁺-5⁺ (2 mM) in DCM ([TBA][PF₆] 0.1 M) solutions recorded at a platinum working electrode (n=0.1 V/s) and Ag/Ag⁺ as internal reference electrode, with Fc/Fc⁺ used as secondary reference by setting its E_{1/2}=0.

FIG. 3 shows results of photoreduction of aryl halides using a helicinium radical of the invention as a photoredox catalyst.

FIG. 4 shows photophysical and electrochemical properties of compound 3.

FIG. 5 illustrates catalytic cycles for (I) C(sp²)-H arylation, and (II) aerobic oxidative hydroxylation.

FIG. 6 illustrates a possible mechanism for red-light-induced ⁿPr-DMQA⁺-catalyzed ATRA.

FIG. 7 is cyclic voltammograms curves of L1-L3 (3 mM) in DCM ([TBA][PF₆] 0.1 M) solutions recorded at a glassy carbon working electrode (n=0.02 V/s).

FIG. 8 shows oxidation and reduction half-wave potential values [V] measured by CV for L1-L3, E, versus the ferrocene/ferrocenium redox couple (Fc/Fc⁺). Redₙ and Oxₙ represent the n successive reduction and oxidation events observed in the tested electrochemical window, respectively.

FIG. 9 shows overlapped absorption spectra and photophysical properties of ligands L1-L3 in acetonitrile.

FIG. 10 shows selected bond lengths/distances (A) and torsion angles (°) of ligand L1 and complexes 2 as well as bond lengths (A) of the ring A of L1 and 2a.

FIG. 11 is CV curves of L1 (3 mM) and complexes 2 (1 mM) in DCM ([TBA][PF₆] 0.1 M) solutions recorded at a glassy carbon working electrode (n=0.02 V/s) along with a table of oxidation and reduction half-wave potential values [V] measured by CV for L1 and 2, E, versus the ferrocene/ferrocenium redox couple (Fc/Fc⁺).

FIG. 12 is CV curves of L3 (3 mM) and complexes 4 (1 mM) in DCM ([TBA][PF₆] 0.1 M) solutions recorded at a glassy carbon working electrode (n=0.02 V/s) along with a table of oxidation and reduction half-wave potential values [V] measured by CV for L3 and 4, E, versus the ferrocene/ferrocenium redox couple (Fc/Fc⁺).

FIG. 13 shows electrochemical response curve of NiCl₂(DME) (1 mM), L1 (1 mM), 2b (1 mM) with acetic acid (50 mM) in CH₃CN ([TBA][PF₆] 0.1 M) solutions recorded at a glassy carbon working electrode (n=0.1 V/s). E, versus the ferrocene/ferrocenium redox couple (Fc/Fc⁺).

FIG. 14 shows electrochemical response curve of NiCl₂(DME) (1 mM), 4b (1 mM) with acetic acid (50 mM) in CH₃CN ([TBA][PF₆] 0.1 M) solutions recorded at a glassy carbon working electrode (n=0.1 V/s). E, versus the ferrocene/ferrocenium redox couple (Fc/Fc⁺).

FIG. 15 shows electrochemical response curve of 2b (1 mM) at various acetic acid concentration (x mM) in CH₃CN ([TBA][PF₆] 0.1 M) solutions recorded at a glassy carbon working electrode (n=0.1 V/s). E, versus the ferrocene/ferrocenium redox couple (Fc/Fc⁺).

FIG. 16 shows electrochemical response curve of 4b (1 mM) at various acetic acid concentration (x mM) in CH₃CN ([TBA][PF₆] 0.1 M) solutions recorded at a glassy carbon working electrode (n=0.1 V/s). E, versus the ferrocene/ferrocenium redox couple (Fc/Fc⁺).

FIG. 17 is overlapped absorption spectra in acetonitrile of ligand L1 and complex 2.

FIG. 18 is overlapped absorption spectra in acetonitrile of ligand L3 and complex 4.

FIG. 19 is a table showing photophysical properties of ligands L1, L3 and complexes 2,4 in acetonitrile.

### DESCRIPTION OF THE INVENTION

The present invention provides a new class of organic-based photocatalysts, such as those can be activated using a low energy electromagnetic radiation. In particular, organic-based PCs of the invention can be activated using electromagnetic radiation wavelength of about 390 nm or greater, typically about 500 nm or greater, and often about 600 nm or greater. As used herein, the terms “about” and “approximately” when referring to a numerical value are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value, typically within one standard deviation.

Some aspects of the invention provide a compound comprising a moiety of the formula:

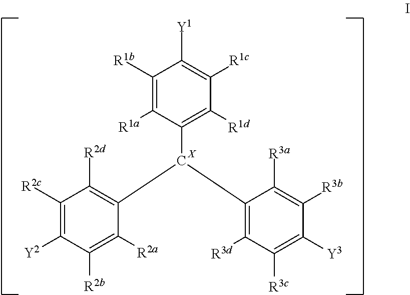

where the moiety of Formula I is a radical, a cation, or a radical dication and “x” indicates “central carbon atom”. With respect to the moiety of Formula I: each of R^{1a}, R^{1b}, R^{1c}, R^{1d}, R^{2a}, R^{2b}, R^{2c}, R^{2a}, R^{3a}, R^{3b}, R^{3c}, and R^{3d} is independently H, halide, haloalkyl (e.g., CF₃), —NR^{a}R^{b} (each of R^{a} and R^{b} is independently H or C₁-C₄ alkyl), C₁-C₁₂ alkyl, C₁-C₄ alkoxy, —NO₂, —CN, —CO₂R (where R is H or C₁-C₄ alkyl), or Ar¹;
or R^{2a} and R^{3d} together form —X¹—;
or R^{1a} and R^{2d} together form —X²—;
or R^{1d} and R^{3a} together form —X³—;
each of X¹, X² and X³ is independently O, NR^{4a}, PR^{4a}, CR^{4a}R^{4b}, or SiR^{4a}R^{4b};
or R^{1a} and R^{1b} together with atoms to which they are attached to form an optionally substituted aromatic ring (e.g., optionally substituted phenyl);
or R^{2c} and R^{2d} together with atoms to which they are attached to form an optionally substituted aromatic ring (e.g., optionally substituted phenyl);
each of Y¹, Y², and Y³ is independently H, OR^{1a}, CR^{5a}R^{5b}R^{5b} NR^{5a}R^{5b} PR^{5a}R^{5b}, NO₂, CN, CF₃, CO₂R, N₃, or Ar¹;
each of R^{4a}, R^{4b}, R^{5a}, and R^{5b} is independently H, halide, haloalkyl (e.g., CF₃), C₁-C₁₂ alkyl, C₁-C₄ alkoxy, —NR^{a}R^{b} (provided at least one of R^{a} and R^{b} is C₁-C₄ alkyl), Ar¹, or a moiety of the formula -L-Z,
where L is a linker (such as an alkylene, ethylene glycol or other glycols, (e.g., propylene glycol) of 2-20 monomeric units); and Z is a heterocyclic species of Formula I (i.e., dimer, trimer, or other oligomers of Formula I), a coordinating group able to bind or complex to a metal ion, or a water-soluble group such as phosphate, phosphite, sulfate or sulfite; and Ar¹ is optionally substituted aryl (e.g., optionally substituted phenyl) having from 0 to 5 substituents, each of which is independently selected from the group consisting of X1, haloalkyl (e.g., CF3), NRaRb, C1-C4 alkyl, and C1-C4 alkoxy, heteroaryl, fused aryl or Ar¹,
   provided when said Formula I is a cation then
(i) when R^{2a} and R^{3d} together form —X¹—; R^{1a} and R^{2d} together form —X²—; and R^{1d} and R^{3a} together form —X³—, then no more than one of X¹, X², and X³ is O;
(ii) when R^{2a} and R^{3d} together form —X¹—, and R^{1a} and R^{2d} together does not form —X²—; and R^{1d} and R^{3a} together does not form —X³—, then at least one of R^{1a}, R^{1b}, R^{1c}, R^{1d}, or Y¹ is not H, halogen, or C₁-C₁₂ alkyl; in some embodiments, Y¹ is NR^{5a}R^{5b}, PR^{5a}R^{5b}, haloalkyl (e.g., CF₃), N₃, or Ar¹;
(iii) at least one of (a) R^{2a} and R^{3d} together form —X¹—; (b) R^{1a} and R^{2d} together form —X²—; or (c) R^{1d} and R^{3a} together form —X³—; and
(iv) when R^{2a} and R^{3d} together form —X¹—; R^{1a} and R^{2d} together form —X²—; and R^{1d} and R^{3a} together form —X³—, then none of X¹, X², and X³ is NR^{4a}.

In some embodiments, the moiety of Formula I is selected from the group consisting of:

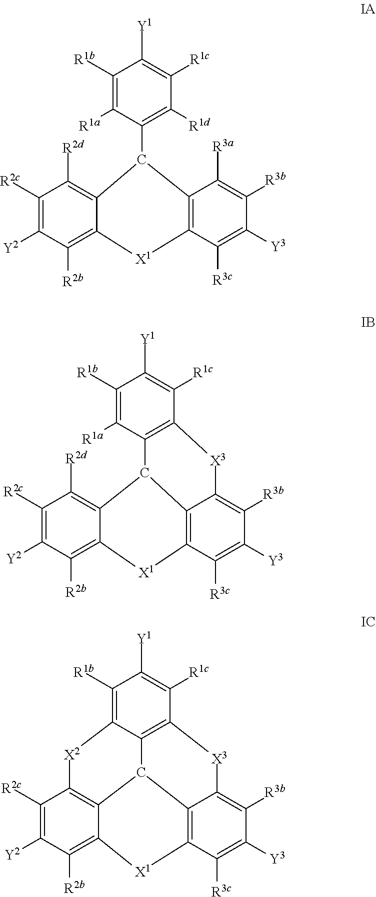

where the moiety of Formulas IA, IB, and IC are radical, cation, or radical dication; and Y¹, Y², Y³, X¹, X², X³, R^{1a}, R^{1b}, R^{1c}, R^{1d}, R^{2b}, R^{2c}, R^{2a}, R^{3a}, R^{3b}, and R^{3c} are those defined herein. As used herein, the terms “those defined above” and “those defined herein” when referring to a variable incorporates by reference the broad definition of the variable as well as any narrower definitions.

In some embodiments, when moiety of Formula I, IA, IB, or IC is a cation. Still in other embodiments, the moiety of Formula I, IA, IB or IC is a radical or a radical dication.

The compounds of the invention can be used in a wide variety of applications including, but not limited to, as efficient photocatalyst using light. Unlike other conventional organic-based PCs, compounds of the invention can be activated using a low energy electromagnetic radiation. Compounds of the invention are also useful in electronic equipment or as solid phase catalysts in organic reactions. Some of the advantages of compounds of the invention are believed to be due at least in part because: (i) they are among the most stable carbocations, radicals, and diradical cations known to date, including under mild acidic or basic aqueous conditions; (ii) the stepwise and temperature dependence of the synthesis allows versatility of using a wide variety of starting materials for synthesis (e.g., by using aliphatic or aromatic amines) and allows formation of unsymmetrical ions; (iii) they can be readily functionalized, e.g., via C—H metal-catalyzed cross-coupling; and (iv) the negative counterions can be exchanged to affect the physical and chemical properties of the salts. Additionally, compounds of the invention are redox active compounds, thereby allowing them to be used in various electronic equipment.

Referring to Formula I, the term “alkyl” refers to a saturated linear monovalent hydrocarbon moiety. Unless the number of carbon atoms is specified, the term “alkyl” typically refers to hydrocarbon moiety having one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2-propyl, tert-butyl, pentyl, and the like. The term “alkoxy” refers to a moiety of the formula —OR, wherein R^{a} is alkyl as defined herein. The term “alkylamino” refers to a moiety of the formula —NHR^{a}, where R^{a} is alkyl as defined herein. The term “dialkyl amino” refers to a moiety of the formula —NR^{a}R^{b}, wherein R^{a} and R^{b} are independently alkyl as defined herein. The terms “aryl” and “aromatic ring” are used interchangeably herein and refer to a monovalent mono-, bi- or tricyclic aromatic hydrocarbon moiety of 6 to 15 ring atoms. Exemplary aryl groups include phenyl, naphthyl, and anthracene. Aryl may optionally be substituted with one or more substituents. When substituted, aryl comprises from one to five, typically, one to four, often one to three, more often one or two, and most often one substituent. Suitable substituent(s) for an aryl group include, but are not limited to, halogen (e.g., Cl, F, I, Br, typically Cl or F), alkyl, haloalkyl, hydroxy, alkoxy, heteroalkyl, cyano, nitro, nitroso, carboxylic acid or esters, etc. When more than one substituent is present in the aryl group, each substituent is independently selected. The term “linker” refers to a moiety having from 1 to 20 atoms in the chain. Typically, the chain atoms include carbon, oxygen, nitrogen, sulfur, and phosphorous, provided oxygen-oxygen bond or nitrogen-nitrogen bond is not present in the chain. Often the linker is a hydrocarbon moiety or an ether moiety. The term “heteroaryl” means a monovalent monocyclic or bicyclic aromatic moiety of 5 to 12 ring atoms containing one, two, or three ring heteroatoms selected from N, O, or S, the remaining ring atoms being C. Exemplary heteroaryl includes, but is not limited to, pyridyl, furanyl, thiophenyl, thiazolyl, isothiazolyl, triazolyl, imidazolyl, oxazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, thiazolyl, isothiazolyl, quinolyl, isoquinolyl, benzimidazolyl, benzisoxazolyl, benzothiophenyl, dibenzofuran, and benzodiazepin-2-one-5-yl, and the like. The term “heteroalkyl” means a branched or unbranched, alkyl moiety as defined herein but where one or more carbon or hydrogen is replaced with a heteroatom (e.g., O, N, S) or where one or more heteroatom-containing substituents is present such as ═O, —OR^{a}, —C(O)R^{a}, —NR^{b}R^{c}, —C(O)NR^{b}R^{c} and —S(O)ₙR^{d} (where n is an integer from 0 to 2, and each of R^{a}, R^{b}, R^{c}, R^{d} is independently hydrogen or alkyl). “Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

It should be appreciated moieties of Formulas I, IA, IB, and IC themselves do not necessarily comprise chemical compounds. Indeed, in an isolable compound, corresponding cations, radicals, dication radicals must be paired with a corresponding moiety that provides electroneutrality and overall non-radical compound. Thus, compounds of the present invention include corresponding anion, radical, or dication radical that provides overall non-radical electroneutral compound (i.e., “corresponding moiety”). The nature of the corresponding moiety is not as important as the carbocation, radical, or dication radical of Formulas I, IA, IB, and IC (collectively simply referred herein as “Formula I”) since the electronic property of compounds of the invention is primarily due to the carbocation, radical, or radical dication of Formula I. Exemplary counter moiety of Formula I include, but are not limited to, any anion including, but not limited to, anionic or radical metal salts, halides (e.g., Cl, F, I, and Br), an anion or a radical derived from organic compounds such as carboxylates, phosphates, sulfates, etc.

In some embodiments, the moiety of Formula I (as well as other compounds disclosed herein) do not require the moiety of the formula -L-Z. In these embodiments, the metal complex can be present in solution as a separate entity to provide catalytic and/or photovoltaic activity. Accordingly, in some embodiments, the moiety of Formula I lacks a moiety of the formula -L-Z altogether.

Yet in other embodiments, Z is a coordinating group. Generally, a coordinating group refers to a moiety that has one or more heteroatoms that is capable of coordinating to a metal complex. Exemplary coordinating groups include heteroaryl, heteroatom functional groups such as phosphates, sulfates, carbonates, hydroxyl group and ethers, thiol and thiol ethers, amines, imines, carboimides, carbamides, etc.

In one particular embodiment, Z is selected from the group consisting of bipyridinyl, pyridinyl, —PR₂, —OPR₂, —NHC, —NR₂, diimine, imine, —OR, —SR, diphosphines, —RNC, —CO₂H, and carboiimine, where each R is independently H, C₁-C₁₂ alkyl or Aryl.

Still in other embodiments, Z is coordinated (i.e., complexed) to a metal complex. As used herein, “metal complex” refers to a central atom or ion (i.e., coordination center) that is a metal or metal ion and has a surrounding array of bound molecules or ions, which are often called ligands or complexing agents. Exemplary metals in a metal complex include, but not limited to, transition metals, lanthanide metals, actinide metals, alkaline metals, and alkaline earth metals. In one particular embodiment, the metal in a metal complex is a transition metal. Still in another embodiment, the metal is a first-row transition metal. In yet another embodiment, the metal is ones which are known to one skilled in the art as being useful in a catalytic reaction, such as but not limited to, Fe, Ni, Pd, Pt, Rh, Co, Au, Cu, Zn, Ru, etc. It should be appreciated that unless the oxidation state of the metal is explicitly stated, the term “metal” includes metal ions. For example, the term metal referring to iron or Fe includes ferric ion (Fe⁺³) and ferrous ion (Fe⁺²). Similarly, the term metal referring to nickel or Ni includes Ni(I) and Ni(II). In one specific embodiment, the metal is selected from the group consisting of Co (II), Ni (II), Fe (II), Au(I), Pd(II), and Rh(I).

As stated herein, the term “metal complex” also includes ligands or complexing agents. Typically, when the metal is a metal ion, the ligand is a corresponding anionic ligand such as, but not limited to, a halide (e.g., Cl⁻, Br⁻, I⁻, F⁻), a carboxylate (e.g., acetate, oxalate, etc.), hydroxide (—OH), an alkoxide, thiolate, cyclopentadiene (“Cp”), alkyl, oxo, as well as other metal counter ion that is known to one skilled in the art. The ligand or complexing agent can also be a neutral specie such as, but not limited to, naphthalene, phosphine, diphosphine, amine, imine, diamine, diimine, NHC (i.e., N-Heterocyclic Carbene), CO, isocyanides, olefins, alkynes, ethers, pyridines, bipyridines, arenes, etc.

When present, the linker L should be of sufficient length to allow the metal complex that is attached to the moiety of Formula I to interact with the central carbon atom (e.g., carbocation or carbo-radical). As used herein, the central carbon atom refers to the carbon atom that is attached to the three aromatic ring moieties in Formula I that is indicated with an “x”. Accordingly, in some embodiments, L is C₁-C₁₀ alkylene, typically C₂-C₈ alkylene, often C₂-C₆ alkylene, more often C₂-C₅ alkylene, and most often C₂ or C₃ alkylene. It should be appreciated that the linkers are not limited to these specific alkylene ranges and examples given herein. In general, the length of L (including alkylene) can vary in length in order to affect the desired interaction between the metal complex and the central carbon. The term “alkylene” refers to a saturated linear divalent hydrocarbon moiety or a branched saturated divalent hydrocarbon moiety. Exemplary alkylene groups include, but are not limited to, methylene, ethylene, propylene, butylene, pentylene, 2-methylpropylene, and the like.

Linkers can also be a C₁-C₁₀ heteroalkylene such as ethers, etc. The term “heteroalkylene” refers to an akylene group as defined herein in which one or more chain atoms or hydrogen atoms are replaced with a heteroatom such as O, N, P, or S. Exemplary heteroalkylenes include, but are not limited to, polyethylene glycol derived heteroalkylenes such as PEG2 (i.e., 2 molecules of ethylene glycol linker), PEG3, 2-methoxyethylene, 2-hydroxyethyl, 2,3-dihydroxypropyl, etc.

Compounds of the invention can be used in a wide variety of applications including, but not limited to, as photocatalysts, as photovoltaic cells, as artificial photosynthesis materials, as organic light-emitting diodes, (“OLEDs”), etc.

Some of the advantages of compounds of the invention include activation of compounds using a much lower energy electromagnetic radiation compared to conventional photocatalysts. In particular, surprisingly and unexpectedly the present inventors have found that compounds of the invention can be activated by wavelength of 390 nm or longer, often 500 nm or longer, and most often 600 nm or longer. In some embodiments, catalytic activity can be observed with electromagnetic radiation having wavelength from about 600 nm to about 1,000 nm, often from about 600 nm to about 800 nm, and more often from about 600 nm to about 700 nm.

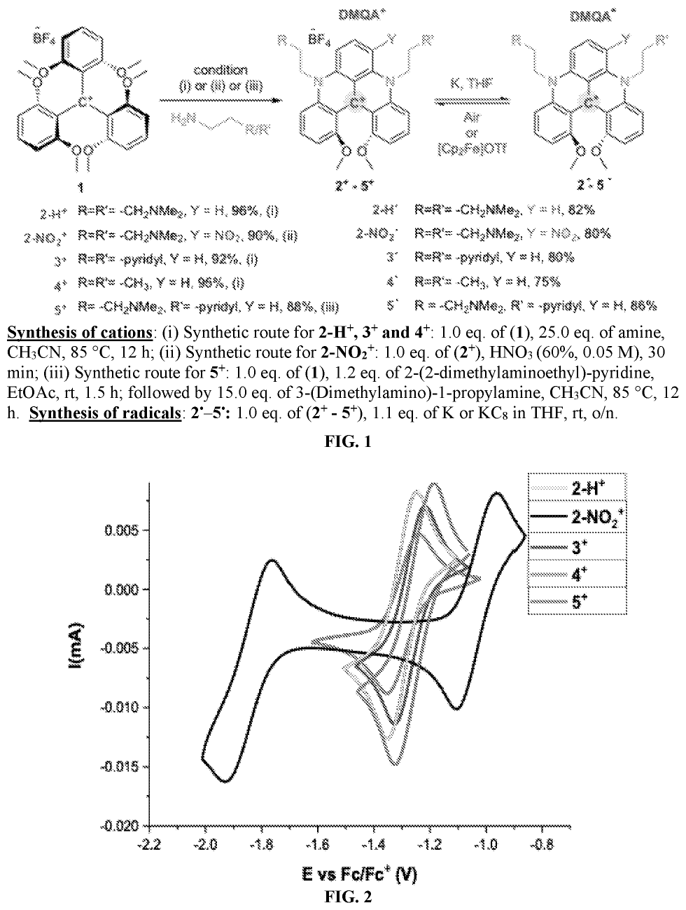

Compounds of the invention can be prepared as shown in FIG. 1, or using the following general synthetic method with appropriately substituted reagents and reaction conditions known to one skilled in the art:

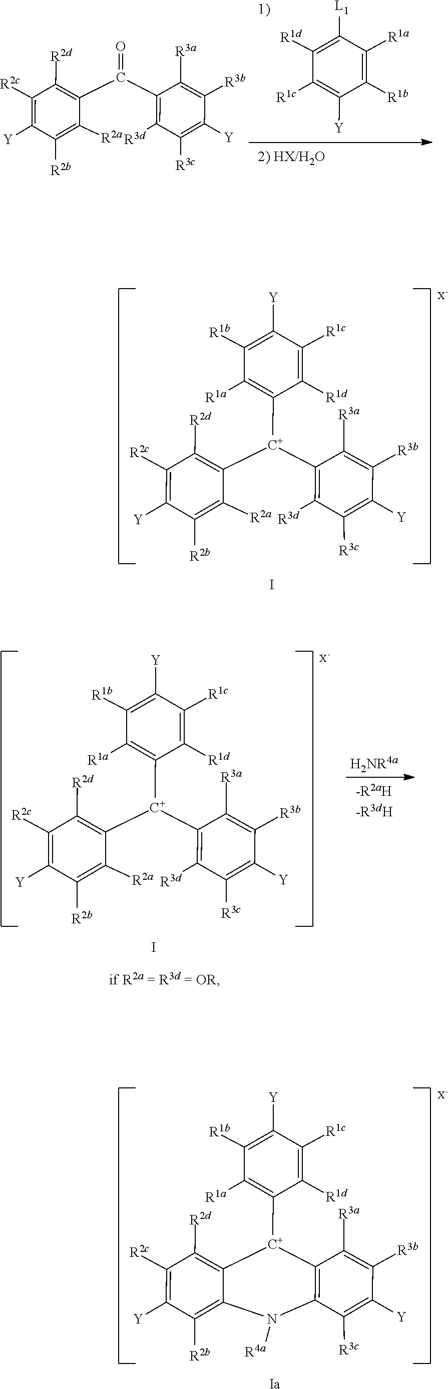

Surprisingly and unexpectedly, the present inventors have discovered that stable radicals and radical dications can also be prepared. Stable radicals are of particular interest to the scientific community for their applications in catalysis, OLEDs and other material applications. In recent years, organic radicals have enjoyed increasing interest due to their involvement in the fast growing field of photocatalysis. During the photoinduced electron transfer (PET) process, an electron transfer occurs between the excited state of an organic photocatalyst and an electrochemically matched substrate, resulting in the formation of an organic radical. In almost all systems, the radical moiety remains an unisolable transient intermediate. Interestingly, it has recently been reported that organic radicals formed in situ by light-induced reduction of their closed-shell photocatalysts counterpart (i.e., perylenediimine PDI and mesityl acridnium salt (i.e., Mes-Acr⁺), respectively) can further absorb light and efficiently catalyze the photoreduction of aryl halide. Despite early reports showing that transient organic radicals such as diarylketyl and diphenylmethyl can undergo photoinduced electron transfer (PET), PDI-. and Mes-Acr. are the only organic radical photocatalyst reported to date.

It was discovered by the present inventors that compounds of the invention are efficient photoinduced electron acceptors and can form helicene radicals as well as radical dications during the photocatalysis of organic transformation using a red light-emitting diode (LED) as a light source. To date, only a few examples of [n]helicene (n=4, 5, 6, 7 indicates the number of fused aromatic rings) radicals have been reported in the literature. Without being bound by any theory, it is believed that the unpaired electron in these systems is stabilized through delocalization over the π-conjugated substituents, and the molecules with a higher number of fused aromatic rings show a higher stability. Thus, helicenes represent the least stable and least studied radicals of this class. To date, only four examples of helicene radicals have been reported, two of which showed limited stability, and one other was never isolated. It is also of note that no X-ray crystallographic structure of any helicene-based neutral radical has been reported to date.

Methods of the invention for producing compounds containing a moiety of formula I provide isolation, single crystal structure, and full characterization of a family of helicine radicals, including, but not limited to, methoxyquinacridine (DMQA.), by ¹H NMR, continuous wave (CW) EPR, electron-nuclear double resonance (ENDOR), cyclic voltammetry, UV-vis absorption spectroscopy, as well as density functional theory (DFT) calculations.

Moreover, the invention provides a wide variety of photocatalytic reactions that can be achieved using compounds of the invention including, but not limited to, the reductive dehalogenation of aryl halides, including a less reactive aryl chloride, under photocatalytic condition using an isolated organic radical of the invention.

Another aspect of the invention provides a method of conducting a photocatalytic reaction, such as those disclosed herein. The method generally includes reacting a two or more substrates in the presence of a photocatalyst compound comprising a moiety of the Formula I. In this aspect of the invention, said moiety of Formula I is a radical, a cation, or a radical dication; each of R^{1a}, R^{1b}, R^{1c}, R^{1d}, R^{2a}, R^{2b}, R^{2c}, R^{2a}, R^{3a}, R^{3b}, R^{3c}, and R^{3d} is independently H, halide, haloalkyl, —NR^{a}R^{b} (each of R^{a} and R^{b} is independently H or C₁-C₄ alkyl), C₁-C₁₂ alkyl, C₁-C₄ alkoxy, —NO₂, —CN, —CO₂R (where R is H or C₁-C₄ alkyl), or Ar¹; or R^{2a} and R^{3d} together form —X¹—; or R^{1a} and R^{2d} together form —X²—; or R^{1d} and R^{3a} together form —X³—; each of X¹, X² and X³ is independently O, NR^{4a}, PR^{4a}, CR^{4a}R^{4b}, or SiR^{4a}R^{4b}; or R^{1a} and R^{1b} together with atoms to which they are attached to form an optionally substituted aromatic ring (e.g., optionally substituted phenyl); or R^{2c} and R^{2d} together with atoms to which they are attached to form an optionally substituted aromatic ring (e.g., optionally substituted phenyl); each of Y¹, Y², and Y³ is independently H, OR^{5a}, CR^{5a}R^{5b}R^{5b}, NR^{5a}R^{5b} PR^{5a}R^{5b}, NO₂, CN, CF₃, CO₂R, N₃, or Ar¹; each of R^{4a}, R^{4b}, R^{5a}, and R^{5b} is independently H, halide, haloalkyl (e.g., CF₃), C₁-C₁₂ alkyl, C₁-C₄ alkoxy, —NR^{a}R^{b} (provided at least one of R^{a} and R^{b} is C₁-C₄ alkyl), Ar¹, or a moiety of the formula -L-Z, where L is a linker (such as a alkylene, ethylene glycol or other glycols, (e.g., propylene glycol) of 2-20 units); and Z is a heterocyclic species of formula I (i.e., dimer, trimer, or other oligomers of formula I), a coordinating group able to bind or complex to a metal ion, or a water-soluble group such as phosphate, phosphite, sulfate or sulfite; and Ar is optionally substituted aryl (e.g., optionally substituted phenyl) having from 0 to 5 substituents, each of which is independently selected from the group consisting of X¹, haloalkyl (e.g., CF₃), NR^{a}R^{b}, C₁-C₄ alkyl, C₁-C₄ alkoxy, heteroaryl, fused aryl, or another Ar¹.

In some embodiments, when said formula I is a cation and (i) when R^{2a} and R^{3d} together form —X¹—; R^{1a} and R^{2d} together form —X²—; and R^{1d} and R^{3a} together form —X³—, then no more than one of X¹, X², and X³ is O; (ii) when R^{2a} and R^{3d} together form —X¹—, then Y¹ is NR^{5a}R^{5b}, PR^{5a}R^{5b}, haloalkyl (e.g., CF₃), N₃, or Ar¹; (iii) at least one of (a) R^{2a} and R^{3d} together form —X¹—; (b) R^{1a} and R^{2d} together form —X²—; or (c) R^{1d} and R^{3a} together form —X³—; and (iv) when R^{2a} and R^{3d} together form —X¹—; R^{1a} and R^{2d} together form —X²—; and R^{1d} and R^{3a} together form —X³—, then none of X¹, X², and X³ is NR^{4a}.

Compounds of the invention can be used in a wide variety of photocatalytic processes including those disclosed herein as well as those processes disclosed in DOI: 10.1021/acs.chemrev.9b00045: Chem. Rev. 2019, 119, 6769-6787; doi.org/10.1021/acscatal.8b03050: ACS Catal. 2018, 8, 12, 12046-12055; doi.org/10.1002/ejoc.201700420: DOI: 10.1021/acs.chemrev.6b00057: Chem. Rev. 2016, 116, 10075-10166; DOI: 10.1021/acs.joc.6b01449 J. Org. Chem. 2016, 81, 6898-6926; and dx.doi.org/10.1021/cr300503r: Chem. Rev. 2013, 113, 5322-5363, all of which are incorporated herein by reference in their entirety.

Stable organic radicals are useful species for developing applications in catalysis as well as material sciences. In particular, helical molecules are of great interest to development and application of novel organic radicals in optoelectronic and spintronic materials. The present inventors have discovered that highly stable neutral quinolinoacridine radicals can be produced by chemical reduction of their quinolinoacridinium cation analogs. There radicals can be used in a wide variety of photocatalysis reactions including, but not limited to, photoreductive dehalogenation of aryl halide under irradiation with a relatively low energy electromagnetic radiation.

Synthesis. The helicenium cations (2⁺-5⁺) were synthesized, for example, using the general procedure shown in FIG. 1. The room temperature ¹H NMR spectra of the carbocations 2-H⁺ and 3⁺-5⁺ are well resolved and sharp. However, the ¹H NMR spectrum of 2-NO₂⁺ shows broad and poorly resolved resonances, suggesting a dynamic exchange. Variable temperature (VT)¹H NMR spectroscopy analysis of 2-NO₂⁺ was conducted over a temperature range of 333-193 K confirming the presence of an equilibrium between two conformers. At 193 K, the two ⁿPr—NMe₂ arms are inequivalent, and the assignment of the six methylenic protons was successfully carried out using low-temperature ¹H-¹H COSY NMR sequence. These data support the presence of a NMe₂-C⁺ interaction with an interconversion activation energy of ΔG^{‡}=12.2 kcal/mol.

Referring again to FIG. 1, the radicals 2.-5. were obtained by reduction of 2⁺-5⁺, respectively, with potassium metal in THE at room temperature overnight. The reaction mixture turned from dark green suspension to a dark magenta solution. The insoluble KBF₄ salt that formed in the process was removed by filtration. THE was removed under vacuum, and the resultant solids were extracted with toluene. Crystallization from a toluene/hexane mixture at −35° C. afforded 2.-5. as dark brown crystals in good yields. The formation of radicals 2.-5. was confirmed by EPR spectroscopy. The resultant radicals are remarkably stable under inert atmosphere at room temperature in both the solid and solution, retaining their color and crystallinity indefinitely (at least, over several months). As used herein, the term “stable” refers to having about 10% or less, typically about 5% or less, and often about 1% or less decomposition under standard conditions (i.e., 25° C. at 1 atm pressure) over at least one (1) month period. The analysis of ¹H NMR suggests that the unpaired electron is delocalized onto the fused heterocyclic rings and that the nature of the chosen pendant arms has little influence on the radical character of the compounds 2.-5..

X-ray diffraction. Slow DCM/hexane layering afforded suitable crystals of the cationic precursors 2-H⁺ and 2-NO₂⁺ for X-ray diffraction (XRD) analysis. In both structures, the steric clash between the MeO groups results in a significant twist between the o-(MeO)-phenyl moieties (2-H⁺: 41.93°, 2-NO₂⁺: 38.37°). These differences in torsion angle between 2-H⁺ and 2-NO₂⁺ are also underlined by the O1-O2 distances, 2.743 Å and 2.659 Å, respectively. As deduced from the VT ¹H NMR results, one of the -nPr-NMe₂ arms in 2-NO₂⁺ is folded over the carbocation center with a C1-N3 distance of 3.194 Å.

The neutral radicals 2-H⁺ and 2-NO₂⁺ were isolated from a concentrated toluene solution by slow diffusion of hexane and analyzed by XRD. The distortion of the o-(MeO)-phenyl groups is more accentuated than for the cationic precursors in 2-H⁺ (45.92°, +3.99°), and particularly in 2-NO₂. (52.05°, +13.680), resulting in similar O1-O2 distances for both complexes (2-H⁺: 2.772 Å and 2-NO₂⁺: 2.773 Å). The increase in C1-C2, C1-C3 and C1-C4 interatomic distances in 2-H⁺ (1.439, 1.444, and 1.446 Å, respectively) relative to the cation 2-H⁺ (1.406, 1.435 and 1.431 Å), indicates an antibonding interaction between C1 and its surrounding atoms. Similarly, the C1-C3 distance in 2-NO₂. (1.429 Å) was elongated when compared to 2-NO₂⁺ (1.413 Å). In contrast, bond distances in 2-NO₂⁺ were shortened compared to 2-NO₂⁺ C1-C2 (1.423 vs 1.431 Å) and C1-C4 (1.438 vs 1.440 Å). This phenomenon is assigned to the electron withdrawing ability of NO₂ resulting in a higher delocalization of the electronic charge.

DFT calculations. Computational models of the radicals 2.-5. were studied using DFT to determine their electronic structure. The calculations revealed a considerable spin population on the central carbon in all radicals. Analysis of the frontier molecular orbitals shows that considerable amount of the normalized wavefunction was located on the central carbon in 2.-5., where about 27% of the SOMO's wavefunction is located in this atom regardless of the nature of alkyl groups in the amino substituent or the addition of the electron withdrawing group NO₂. A significant change, however, was observed in comparing the SOMO energy where 2-NO₂. appear significantly stabilized by the electron withdrawing nitro group compare to 2-H..

EPR, ENDOR, Measurements: The EPR spectra of 2-H. and 3.-5. in liquid toluene solutions represent a Gaussian line centered at g≈2.003, with the width of about 0.76 mT and a poorly resolved hyperfine structure with the splitting of 0.088 mT. The EPR spectrum of 2-NO₂. has the same g-factor and width, but the hyperfine structure is unresolved. The ¹H ENDOR spectra of 2-H. and 3.-5. show three pairs of lines ((a,a′), (b,b′), and (c,c′), located at the frequencies of v_{H}±a_{H}/2, where v_{H} is the proton Zeeman frequency and a_{H} is the hyperfine interaction (hfi) constant. The hfi constants estimated for each pair of ENDOR lines are: |a_{Ha}|≈7.1 MHz (for a,a′ lines), |a_{Hb}|≈2.3 MHz (for b,b′ lines), and |a_{Hc}|≈0.65 MHz (for c,c′ lines).

For 2-NO₂., the hydrogen atom is substituted by the NO₂ group, which results in a significant conformational distortion of the aromatic ring structure and a reorientation of the CH₂ group in the vicinity of NO₂. The accompanying changes in the ENDOR spectrum are a ˜30% decrease of the relative intensity of (b,b′) lines and appearance of (d,d′) and (e,e′) lines corresponding to a_{H}=6.05 and 5.37 MHz, respectively. Since the DFT calculation for 2-NO₂. results in essentially the same distribution of spin populations as in 2-H. and 3.-5., these hfi constants were tentatively assigned to the protons of the reoriented CH₂ group.

Electrochemistry: As shown in FIG. 2, the electrochemical behavior of 2.-5. was investigated by cyclic voltammetry. Under reductive conditions, a fully reversible event is observed around E_{1/2}=−1.25 V in 2⁺-5⁺, which corresponds to the reduction of DMQA⁺ to DMQA.. This event is observed at a lower potential for 2-NO₂⁺ (−1.0 V), consistent with a more electron deficient scaffold due to the presence of the NO₂ group. For 2-NO₂⁺, a second reversible reduction was observed at E_{1/2=-1.8} V, while for 2-H⁺, 3⁺-5⁺, an irreversible reduction event is found at E_{1/2}=−2.3 V, corresponds to reduction of DMQA. to DMQA⁻.

UV-Vis spectroscopy. The UV-visible spectra of the cations and radicals (2-5) were studied to understand the electronic transitions. All compounds, cations and radicals, possess three distinct absorption features in the visible regions, with the radical absorption being blue shifted from their cation analogs. The blue shift indicates a reduced involvement of the heteroatoms in the molecular framework as well as a remarkably decreased conjugation. The introduction of the electron withdrawing NO₂ group induces a moderate hypsochromic shift of the lower energy transition and an increase of molar extinction coefficient for the high energy transition in both 2-NO₂⁺ and 2-NO₂. compare to 2-H⁺ and 2-H. respectively.

Upon exposure to air, the absorption spectrum of the radical 2-H. and 3.-5. slowly (within hours) evolves to that of the cationic analog. Unlike most organic radicals, these DMQA radicals do not appear to undergo oxygen insertion or dimer formation. Instead, a clean reversible oxidation to DMQA⁺ is observed. The 2-H. showed the half-life time (t_{1/2}) of about 26 min, 51 min for 3., 54 min for 4., and 44 min for 5.. The 2-NO₂. radical, however, shows a less selective oxidation and a significantly longer t_{1/2} of about 210 min. The reduced reactivity of 2-NO₂. is assigned to the inductive effect of the electron-withdrawing —NO₂ groups.

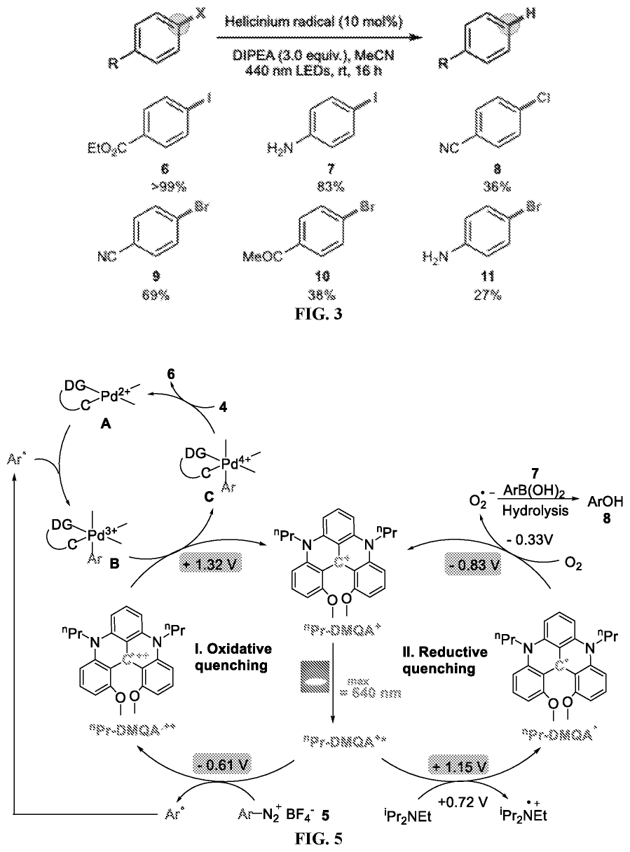

Photocatalysis. The photocatalytic potential of 2-H. was investigated using photo reductive dehalogenation of aryl halides at room temperature in the presence of 10 mol % 2-H. and DIPEA (3.0 eq.) under blue (FIG. 3) or green LED light. The desired reductive dehalogenation of aryl iodides were obtain with high yield. Photoreductive debromination and dechlorination were also successful and provided a moderate to good yield. Photoreduction of 4-iodoaniline using the cationic analog 2-H⁺ was successful, yet with significantly lower yield (42% vs 83%).

Low energy electromagnetic mediated photoredox catalytic reactions. Compounds of the invention can be used as organic-based photoredox catalyst. Unlike conventional organic-based PCs, compounds of the invention can be used as both photoreduction and photooxidation catalysts. Furthermore, compounds of the invention can be activated in the presence of low-energy electromagnetic radiation. In some embodiments, compounds of the invention are used as photocatalysts using a low-energy electromagnetic radiation, e.g., red light. In one particular embodiment, compounds of the invention are used as PCs using electromagnetic radiation having wavelength of about 500 nm or higher, typically about 550 nm or higher, and often about 600 nm or higher. In one specific embodiment, red light (e.g., λₘₐₓ=640 nm) is used to activate photocatalytic activity of compounds of the invention.

It has been discovered that compounds of the invention can catalyze different red-light-mediated reactions including, but not limited to, dual transition-metal/photoredox-catalyzed C—H arylation and intermolecular atom transfer radical addition (ATRA) through oxidative quenching, affording the desired products in up to 93% yield. Moreover, photooxidation properties of compounds of the invention have been demonstrated in the successful applications including, but not limited to, in red-light-induced aerobic oxidative hydroxylation of arylboronic acids and benzylic C(sp3)-H oxygenation through reductive quenching. In some cases, the product yield in the range of 41-92% can be achieved. As used herein, the term “red-light” refers to electromagnetic radiation having the wavelength of from about 600 nm to about 750 nm, typically from about 600 nm to about 700 nm, and often from about 620 nm to about 700 nm.

Photoredox catalysts can be used in a wide variety of chemical reactions. Given the intrinsic disadvantages of transition-metal (TM) catalysts such as high cost, low sustainability and potential toxicity, there has been a large effort in developing organic-based PCs that are inexpensive and environmentally friendly alternatives to TM catalysts. Despite tremendous research, conventional organic-based PCs have many disadvantages, such as being only useful in reductive quenching, having a narrow redox window, being pH dependent, being susceptible to bleaching, and/or requiring high-energy blue, green or white light for their activity. Use of high-energy blue, green or white light is particular problematic in photocatalyst reactions as these high-energy electromagnetic radiations are potentially hazardous to health (e.g., photooxidative damage to retina) and are also more prone to induce undesired products due to their high energy nature.

Compounds of the invention overcomes many of these disadvantages of the conventional organic-based PCs. In particular, compounds of the invention can be activated by red light (e.g., 600-700 nm), which has a significantly low energy thereby causing less side reactions, low health risks and is naturally abundant from sunlight. More importantly, unlike high-energy blue or green lights, red light can also penetrate turbid media.

As an illustrative example, Scheme 1 below shows the photoredox properties of [ⁿPr-DMQA⁺][BF₄⁻] (3) under low-energy red light, and its reactivity in a wide range of red-light-mediated reactions including both reductive and oxidative quenching.

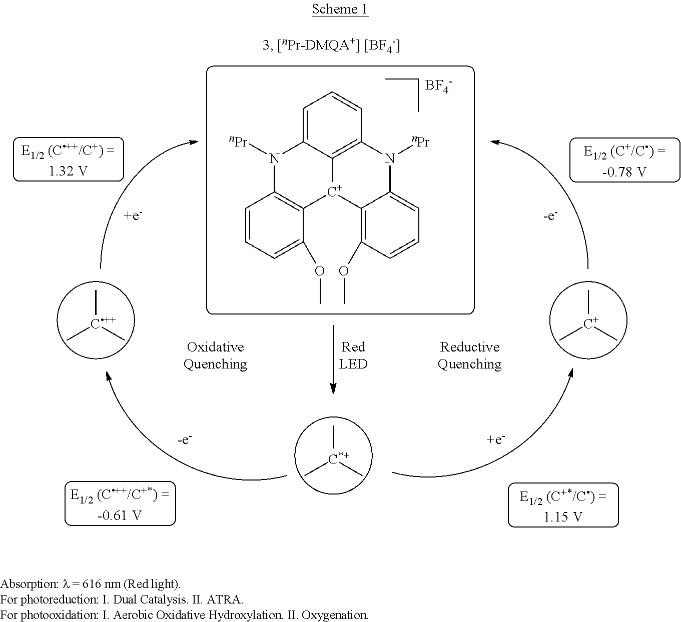

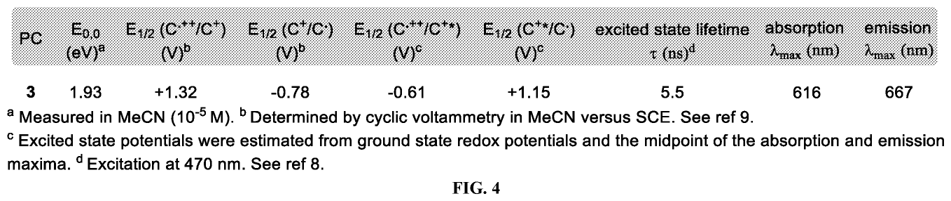

Calculation of the excitation energy (E_{0,0}), excited state oxidation (E_{1/2} (C.⁺⁺/C⁺*)) and reduction potentials (E_{1/2}(C⁺*/C.)) based on the absorption and emission spectra and cyclic voltammetry data for compound 3 are shown in FIG. 4. As illustrated in Scheme 1, compound 3 possesses moderate ground state oxidation and reduction potentials E_{1/2}(C.⁺⁺/C⁺)=+1.32 V and E_{1/2}(C⁺/C.)=−0.78 V vs. the saturated calomel electrode (SCE), as well as moderate excited state oxidation and reduction potentials E_{1/2}(C.⁺⁺/C⁺*)=−0.61 V and E_{1/2}(C⁺*/C.)=+1.15 V. These features render 3 a mild reductant and oxidant whether a reductive or oxidative quenching is involved during photocatalysis. Moreover, the excited state lifetime (τ=5.5 ns) is also comparable to other commonly used organic PCs. In particular, the peak absorption (λₘₐₓ) is 616 nm, which shows compound 3 can be readily activated using a low-energy red light.

Given E_{1/2}(C.⁺⁺/C⁺*)=−0.61 V and E_{1/2}(C.⁺⁺/C⁺)=+1.32 V for 3, a red-light-mediated dual Pd/ⁿPr-DMQA⁺-catalyzed C(sp²)-H arylation using aryldiazonium salts was conducted. The reaction between 1-([1,1′-biphenyl]-2-yl)pyrrolidin-2-one (4a) and benzenediazonium tetrafluoroborate (5a) proceeded smoothly in the presence of Pd(OAc)₂ and 3 under red LED (λₘₐₓ=640 nm), affording the desired product 6aa in 95% NMR yield after 4 hours. In the absence of 3, red light, or Pd(OAc)₂, significantly lower yields (≤25%) of 6aa was observed.

TABLE 1
Red-Light-Mediated Dual Pd/ⁿPr-DMQA⁺-Catalyzed C(sp²)-H Arylation

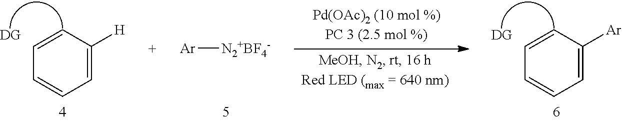

| Entry^{a} | Substrate 4 | Substrate 5 | Product 6; yield^{b} |
|---|---|---|---|
| 1 | 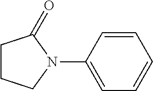 [SMILES: O=C1CCCN1c1ccccc1] | 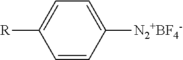 | 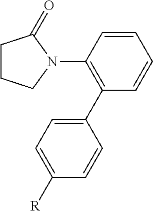 |
| | 4a | 5a: R = H | 6aa: R = H, 93%^{c} |
| | | 5b: R = Cl | 6ab: R = Cl, 92%^{c} |
| | | 5c: R = OMe | 6ac: R = OMe, 86% |
| 2 | 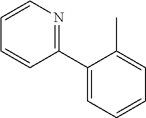 | 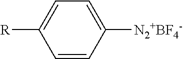 | 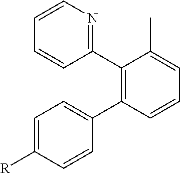 |
| | 4b | 5a: R = H | 6ba: R = H, 73% |
| | | 5b: R = Cl | 6bb: R = Cl, 68% |
| | | 5c: R = OMe | 6bc: R = OMe, 60% |
| 3 | 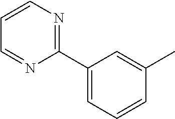 | Ph—N₂⁺BF₄⁻ | 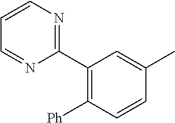 [SMILES: Cc1ccc(-c2ccccc2)c(-c2ncccn2)c1] |
| | 4c | 5a | 6ca: 57% |
| 4 | 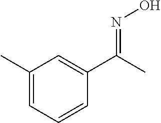 | Ph—N₂⁺BF₄⁻ | 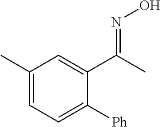 [SMILES: C/C(=N\O)c1cc(C)ccc1-c1ccccc1] |
| | 4d | 5a | 6da: 64% |

^{a}The reaction was conducted with 4 (0.2 mmol), 5 (0.8 mmol), Pd(OAc)₂ (10 mol %), and PC 3 (2.5 mol %) in MeOH (2.0 mL).
^{b}Isolated yield.
^{c}The reaction was ran for 4 h.

Table 1 shows this red-light-mediated dual Pd/ⁿPr-DMQA⁺-catalyzed C(sp²)-H arylation is applicable to a wide variety of substrates. By using 4a as the model substrate, different electron-neutral, electron-deficient and electron-rich aryldiazonium salts 5a-5c were examined. The reactions proceeded smoothly, delivering the desired products 6aa-6ac in 86-93% (Table 1, entry 1). Substrate 4b containing pyridine as the directing group (DG) was well-tolerated as well, furnishing the corresponding coupling products 6ba-6bc in 60-73% yields by reacting with 5a-5c (Table 1, entry 2). In addition, the desired C—H arylated product 6ca could also be obtained in 57% yield when 4c with pyrimidine as the DG reacted with 5a (Table 1, entry 3). The oxime derivative 6da was isolated in 64% yield when substrate 4d was tested with 5a (Table 1, entry 4). Notably, all the products 6 were obtained in relatively high isolated yields. Furthermore, experiments in the absence of 3, red light or Pd(OAc)₂ were also conducted, affording 6 in much lower yields for all substrates. The successful application of 3 in this red-light-mediated C—H arylation shows that it is capable of use in oxidative quenching during photocatalysis.

Compounds of the invention can also be used in red-light-mediated reductive quenching. As an illustrative example, the same compound 3 was used in photo-induced aerobic oxidative hydroxylation of arylboronic acids. A representative reaction involves the oxidation of ⁱPr₂NEt (DIPEA) by an excited state PC* to form the radical cation ⁱPr₂NEt.⁺ (ⁱPr²NEt/ⁱPr²NEt.⁺=+0.72 V vs. SCE) and the reduction of O₂ by the PC.⁻ to generate the superoxide radical anion O₂.⁻(O₂/O₂.⁻=−0.33 V). With the excited state reduction potential (E_{1/2}(C⁺*/C.)) at +1.15 V and ground state reduction potential (E_{1/2}(C⁺/C.)) at −0.78 V, the present inventors believed that compound 3 would be competent to serve as an efficient PC for this reaction. Experiments with phenol 8a was achieved in 87% NMR and 83% isolated yield when phenylboronic acid 7a was treated with DIPEA and 3 in the presence of air and red light for 24 hours in DMF (Table 2). In the absence of PC or red light, little or no conversion was observed (Table 2).

TABLE 2
Optimization and control experiments of ⁿPr-DMQA⁺-catalyzed aerobic
oxidative hydroxylation of arylboronic acids

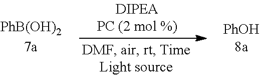

| Entry^{a} | PC | Light Source | Time (h) | Yield (%)^{b} |
|---|---|---|---|---|
| 1 | 3 | Red LED (640 nm) | 18 | 77 |
| 2 | 3 | Red LED (640 nm) | 24 | 87 (83)^{c} |
| 3 | 3 | Red LED (640 nm) | 28 | 88 |
| 4 | — | Red LED (640 nm) | 28 | 9 |
| 5 | 3 | — | 28 | n.d. |

^{a}The reaction was conducted with 7a (0.5 mmol), DIPEA (1.0 mmol), and 3 (2 mol %) in DMF (5.0 mL) under air at rt.
^{b}NMR yield by using 1,3,5-trimethoxybenzene as internal standard.
^{c}isolated yield.

Under the standard reaction conditions, the substrate scope was explored, and the results are presented in Table 3. A wide range of arylboronic acids 7 with diverse useful functional groups such as halide (7c), nitrile (7d), aldehyde (7e), ester (7f), carboxylic acid (7g) and nitro (7k) were well-tolerated, producing the desired phenols 8a-8l. The substitution pattern on the phenyl ring or electronic properties of 7 didn't have much influence over the reaction outcome. For example, 8b-8g was isolated in 41-87% yield when 7b-7g with different electron-donating groups (EDG) or electron-withdrawing groups (EWG) at para position were examined. Substrates 7h-7k with diverse substituents at ortho or meta position also provided the corresponding phenols 8h-8k in 55-73% yields. 2-Naphthylboronic acid 71 was also suitable for this oxidative hydroxylation, providing naphthalen-2-ol 8l in 65% yield. This red-light-induced ⁿPr-DMQA⁺-catalyzed aerobic oxidative hydroxylation shows that 3 is an efficient PC for photocatalysis involving reductive quenching.

TABLE 3
ⁿPr-DMQA⁺-Catalyzed Aerobic Oxidative Hydroxylation of Arylboronic
Acids under Red Light

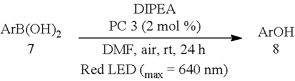

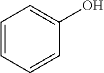

8a, 83%

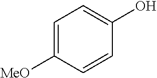

8b, 71%

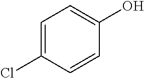

8c, 81%

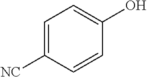

8d, 87%

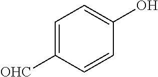

8e, 66%

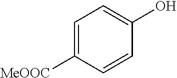 [SMILES: COC(=O)c1ccc(O)cc1]

8f, 80%

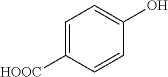

8g, 41%

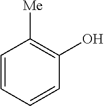

8h, 72%

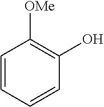

8i, 55%

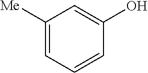

8j, 67%

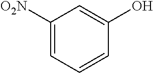

8k, 73%

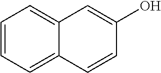 [SMILES: Oc1ccc2ccccc2c1]

8l, 65%
^{a}The reaction was conducted with 7 (0.5 mmol), DIPEA (1.0 mmol), and 3 (2 mol %) in DMF (5.0 mL) under red LED (640 nm) in the presence of air at rt for 24 h.
Isolated yields were shown.

To further illustrate the generality of 3 as a PC, several other red-light-induced transformations were investigated (Scheme 2). As another example of photocatalysis involving in reductive quenching and O₂.⁻, visible-light-mediated aerobic benzylic C(sp³)-H oxygenation using oxygen as the oxidant was extensively studied. When a tertiary amine 9 was treated with 1,8-diazabicyclo[5.4.0]undec-7-ene (DBU) and 3 in the presence of air and red light for 20 hours in DMF, the amide 10 was isolated in 92% yield (Scheme 2a). Atom transfer radical addition (ATRA) of organic halides to olefins serves as an atom-economical approach of simultaneously forming C—C and C—X bonds, and one of the most effective way to accomplish it is photocatalysis through oxidative quenching. As shown in Scheme 2b, in the presence of LiBr and 3a, a red-light-induced intermolecular ATRA between 4-nitrobenzyl bromide 11 and styrene 12 was realized, affording the desired adduct 13 in 59% yield. Compound 3 could also act as a dual gold/photoredox-catalyzed C(sp)-H arylation of terminal alkyne 14 with 5a, providing the desired product 15 in 62% yield (Scheme 2). Control experiments in the absence of 3, red light, or other reagents such as DBU, LiBr or Au(PPh₃)Cl were also performed. The reduced yields of 10, 13, and 15 further supports the photocatalytic roles of PC 3 and red light for these reactions.

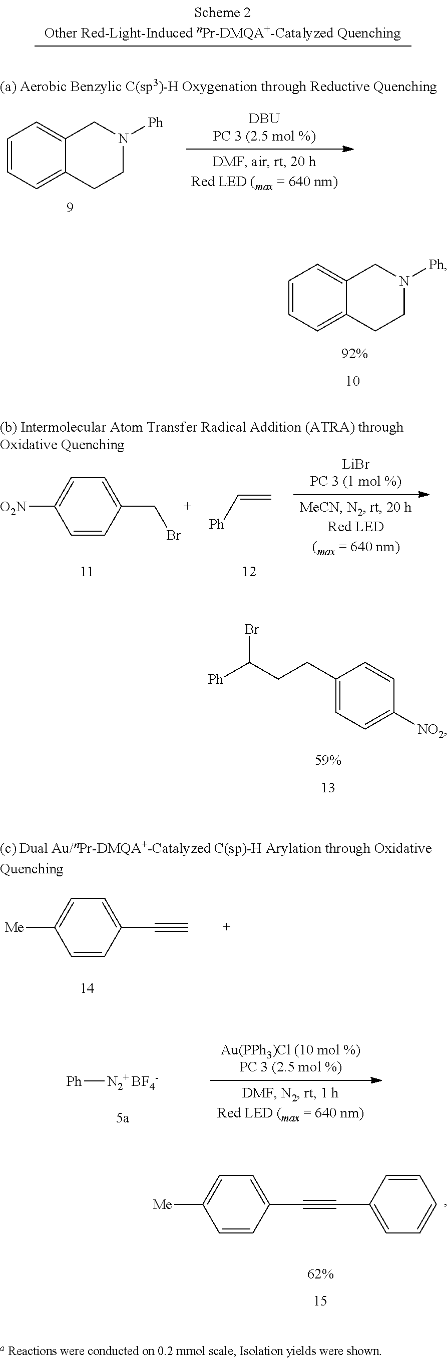

Without being bound by any theory, two representative catalytic cycles involving in oxidative or reductive quenching are illustrated in FIG. 5. Route I in FIG. 5 shows the possible mechanism for dual Pd/ⁿPr-DMQA⁺-catalyzed C(sp²)-H arylation. Firstly, photoexcitation of 3 generates ⁿPr-DMQA⁺*, which reduces aryldiazonium 5 to form an aryl radical and ⁿPr-DMQA.⁺⁺ through oxidative quenching. Then, addition of the aryl radical to the Pd(II) intermediate A (generated by C—H activation of substrate 4) affords the Pd(III) species B, followed by an one-electron oxidation with ⁿPr-DMQA.⁺⁺ to regenerate ⁿPr-DMQA⁺ and form a Pd(IV) intermediate C. Reductive elimination of the intermediate C then produces the arylated product 6. Mechanism for ⁿPr-DMQA⁺-catalyzed aerobic oxidative hydroxylation is illustrated in route II. ⁱPr₂NEt is first oxidized to an ammonium radical cation by the excited state of 3, along with a helicene radical ⁿPr-DMQA. through reductive quenching. Then, the helicene radical further reacts with oxygen to regenerate 3 and form an O₂.⁻. Follow-up oxidative attack onto substrates 7 and hydrolysis afford phenols 8.

Compounds of the invention, e.g., a helical carbenium organic PC—[ⁿPr-DMQA⁺][BF₄⁻] (3), features both photoreductions and photooxidations in the presence of low-energy red light. The role of compounds of the invention as an efficient PC in oxidative and reductive quenching were assessed on a wide range of photoredox reactions, including TM/ⁿPr-DMQA⁺-catalyzed C—H arylations and intermolecular ATRA (oxidative quenching), as well as aerobic oxidative hydroxylation of arylboronic acids and benzylic C(sp³)-H oxygenation (reductive quenching). Many diverse substrates with different DGs have been well-tolerated for the red-light-mediated dual Pd/ⁿPr-DMQA⁺-catalyzed C(sp²)-H arylation, delivering the desired products 6 in up to 93% yield. Red-light-induced ⁿPr-DMQA⁺-catalyzed aerobic oxidative hydroxylation has also been examined with many different arylboronic acids, furnishing the corresponding phenols 8 in 41-87% yields. The success in catalyzing these reactions under red light have proven the role of compounds of the invention as a versatile organic PC.

One particular aspect of the invention provides a carbenium comprising a coordinating group that is capable of coordinating to a metal complex. Such a compound can be used in a wide variety of applications including, but not limited to, as homogeneous catalysts (e.g., photocatalysts) in chemical reactions, as redox compounds in photovoltaic cells, as compounds in artificial photosynthesis applications, etc. In one particular embodiment, compounds of the invention comprise a conjugated heterocyclic carbenium ion that is suitable to bind to or is bound to a metal complex.

Compounds of the invention are useful in electronic equipment as well as catalysts in various organic reactions. One particular aspect of the invention provides a compound comprising a carbocation of the formula:

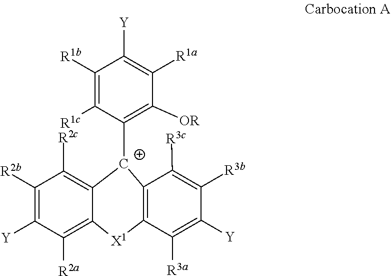

where R is C₁-C₁₂ alkyl or Aryl; X¹ is O, NR^{4a}, PR^{4a}, CR^{4a}R^{4b}, or SiR^{4a}R^{4b}; each of Y is independently H, OR^{5a}, NR^{5a}R^{5b}, PR^{5a}R^{5b}, NO₂, CN, haloalkyl (e.g., CF₃), N₃ CO₂R, or Ar¹; each of R^{1a}, R^{1b}, R^{1c}, R^{2a}, R^{2b}, R^{2c}, R^{3a}, R^{3b}, and R^{3c}, is independently H, halide, CF₃, NH₂, C₁-C₁₂ alkyl, C₁-C₄ alkoxy, C₁-C₄ alkylamino, C₁-C₄ dialkyl amino, NO₂, CN, CO₂R, or Ar¹; each of each of R^{4a}, R^{4b}, R^{5a}, and R^{5b} is independently H, halide, CF₃, C₁-C₁₂ alkyl, C₁-C₄ alkoxy, C₁-C₄ alkylamino, C₁-C₄ dialkyl amino, or Ar¹; or R^{1c} and R^{2c} together form —X²—; or OR and R^{3c} together form —X³—; each of X² and X³ is independently O, NR^{4a}, PR^{4a}, CR^{4a}R^{4b}, or SiR^{4a}R^{4b}; or R^{1b} and R^{1c} together with atoms to which they are attached to form a phenyl; or R^{2b} and R^{2c} together with atoms to which they are attached to form a phenyl; Ar¹ is optionally substituted phenyl having from 0 to 5 substituents, each of which is independently selected from the group consisting of X¹, CF₃, NH₂, C₁-C₄ alkyl, C₁-C₄ alkoxy, C₁-C₄ alkylamino, and C₁-C₄ dialkyl amino; and wherein at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, wherein L is a linker; and Z is a coordinating group that is capable of coordinating to a metal complex or is coordinated to a metal complex. Z can also be a functional group that can be used to attached to a surface of a solid substrate.

Some of the advantages of compounds of the invention comprising carbocation A are believed to be due at least in part because: (i) they are among the most stable carbocations to date, including under mild acidic or basic aqueous conditions; (ii) the stepwise and temperature dependence of the synthesis allows versatility of using a wide variety of starting materials for synthesis (e.g., by using aliphatic or aromatic amines) and allows formation of unsymmetrical ions; (iii) they can be readily functionalized, e.g., via C—H metal-catalyzed cross-coupling; and (iv) the negative counterions can be exchanged to affect the physical and chemical properties of the salts. Additionally, compounds comprising carbocation A are redox active compounds, thereby allowing them to be used in various electronic equipment.

In some embodiments, carbocations of Formula A do not require the moiety of the formula -L-Z. In these embodiments, the metal complex can be present in solution as a separate entity to provide catalytic and/or photovoltaic activity. Still in other embodiments, carbocation of Formula A lacks a moiety of the formula -L-Z altogether.

In some embodiments, the carbocation or carbenium is of the formula:

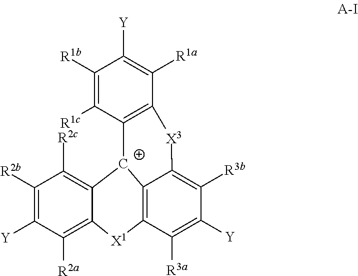

wherein Y, X¹, X³, R^{1a}, R^{1b}, R^{1c}, R^{2a}, R^{2b}, R^{2c}, R^{3a}, and R^{3b} are as defined herein.

Yet in another embodiment, the carbenium is of the formula:

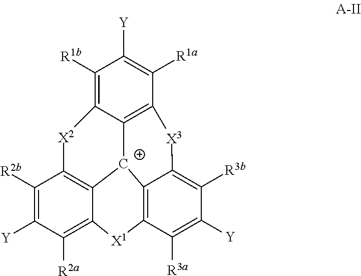

wherein Y, X¹, X², X³, R^{1a}, R^{1b}, R^{2a}, R^{2b}, R^{3a}, and R^{3b} are as defined herein.

Still in another embodiment, X¹ is NR^{4a}. In some instances within this embodiment, R^{4a} is the moiety of the formula -L-Z.

Still in other embodiments, Z is a coordinating group. Generally, a coordinating group refers to a moiety that has one or more heteroatoms that is capable of coordinating to a metal complex. Exemplary coordinating groups include heteroaryl, heteroatom functional groups such as phosphates, sulfates, carbonates, hydroxyl group and ethers, thiol and thiol ethers, amines, imines, carboimides, carbamides, etc. “Heteroaryl” refers to a monovalent monocyclic or bicyclic aromatic moiety of 5 to 12 ring atoms containing one, two, or three ring heteroatoms selected from N, O, or S, the remaining ring atoms being C. The heteroaryl ring can optionally be substituted with one or more substituents. More specifically the term heteroaryl includes, but is not limited to, pyridyl, furanyl, thiophenyl, thiazolyl, isothiazolyl, triazolyl, imidazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

In one particular embodiment, Z is selected from the group consisting of bipyridinyl, pyridinyl, —PR₂, —OPR₂, —NHC, —NR₂, diimine, imine, —OR, —SR, diphosphines, —RNC, —CO₂H, and carboiimine, where each R is independently H, C₁-C₁₂ alkyl or Aryl.

Yet in other embodiments, each of R^{1c}, R^{2c}, and R^{3c} is independently C₁-C₄ alkoxy. In one particular embodiment, R^{1c}, R^{2c}, and R^{3c} are methoxy.

Still in other embodiments, Y, R^{1a}, R^{1b}, R^{2a}, R^{2b}, R^{3a}, and R^{3b} are H.

Another aspect of the invention provides a photocatalyst composition comprising a conjugated heterocyclic carbenium-metal complex of Formula A in which Z is a coordinating group that is coordinated to a metal complex.

In one particular embodiment, the conjugated heterocyclic carbenium-metal complex is of the formula A-I.

Still in another embodiment, the conjugated heterocyclic carbenium-metal complex is of the formula A-II.

In another aspect of the invention, Z in compound of Formula I is a functional group that is attached to a surface of a solid substrate. In this manner, a solid substrate comprising a carbocation of Formula A is provided. Functional groups that can be used to attach to a surface of a solid substrate depends on the solid substrate material. For example, for Au, often thiol group is used to attach to gold surface, and for silica based solid substrate (e.g., a glass substrate), often amine or silane groups are typically used. Other suitable functional groups that can be used in other solid substrates are well known to one skilled in the art.

In some embodiments, the carbocation attached to a solid substrate surface is of the formula A-I with Z being the coordinating group that is coordinated to a metal complex. Still in other embodiments, the carbocation is of the formula A-IT with Z being the functional group that is used to attach to the solid substrate surface.

Still in other embodiments, the carbocation that is attached to a solid substrate surface can also include a linker and a metal complex. In particular, the carbocation can include a moiety of the formula -L′-Z′, where L′ is linker as defined for L and Z′ is a metal complex as defined for Z. In this manner, the carbocation is attached to a solid substrate via -L-Z moiety and also has at least one linker that is coordinated to a metal complex via -L′-Z′ moiety.

Compounds of the invention can be used in a wide variety of applications including, but not limited to, as photocatalysts, as photovoltaic cells, as artificial photosynthesis materials, etc.

Compounds of the invention comprising a carbocation of Formula A can be prepared as illustrated in scheme A below. By using different starting materials with different substituents on the phenyl ring(s), one can readily prepare a wide variety of different carbocation of Formula A. While not specifically shown in Scheme A below, a further reaction with additional nPr-NH₂ or other -L-Z moiety with provide an angulenium compound (i.e., compound of Formula A-II). Thus, scheme A illustrates a general method for producing acridiniums (e.g., carbocation of Formula A where neither R^{1c} and R^{2c} together nor —OR and R^{3c} together form —X²— or —X³—, respectively), heliceniums (carbocation of Formula A-I) as well as anguleniums (carbocation of Formula A-II).

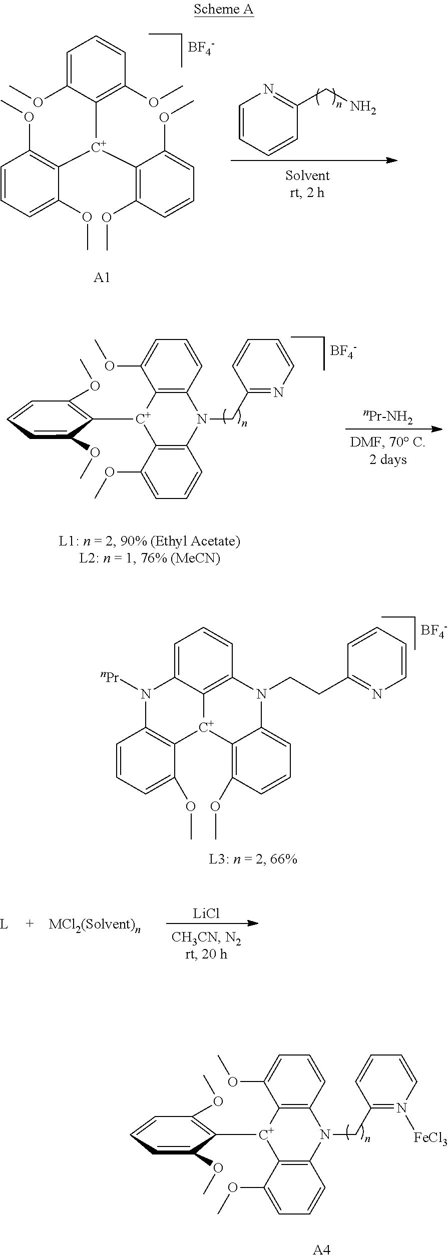

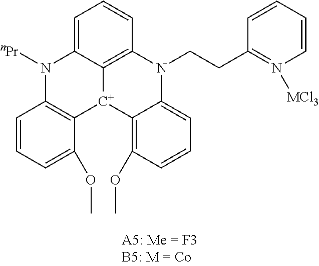

In some embodiments, catalysts of the invention are activated by wavelength of 450 nm or less.

Compounds of the invention can be used as photocatalysts in various organic reactions. The type of organic reaction that can be achieved depends on the metal complex that is attached to the compound of the invention. For example, as shown in Table A, using iron metal complex allows 1,2-radical addition.

The photocatalysis reaction was conducted in a glove box by adding to a Schlenk flask compound 1 (0.2 mmol), compound 2 (1.0 mmol), catalyst (0.006 mmol) and MeCN (2 mL). After the addition of reagents in to the Schlenk flask, the reaction was removed from the glove box and stirred under an inert atmosphere in the presence of blue LEDs (467 nm). When 2.0 equivalent of LiBr was added to the reaction, the yield increased from 12% to 81%. Thus, in some embodiments, an additional reaction enhancing reagent can be added.

As can be seen in Table A, without photoactivation (e.g., no blue LED), no reaction was observed, while ligand L3 and Co-complex 5b gave ˜30% and 8%, respectively. For FeCl₂.(TIF)_{1.5} (entry 10, Table 1) and FeCl₃ (entry 12) no product was observed in the absence of LED. With LEDs, yield of ˜28% and 23%, respectively, was observed. As can be clearly seen in Table A, at best only a trace product was observed when no catalyst was added.

TABLE A
Photocatalysis Reaction

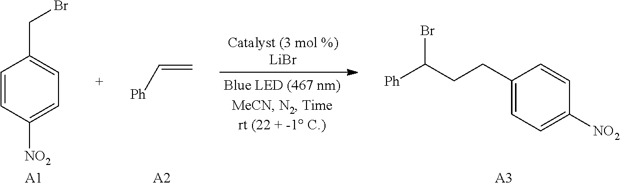

| Entry^{a} | Catalyst | Time [h] | Yield [%]^{b} |
|---|---|---|---|
| 1 | A5 | 13 | ~81 |
| 2 | A5(No LiBr) | 13 | ~12 |
| 3 | A5(No LED) | 13 | N. R. |
| 4 | A5(No LED)^{c} | 13 | N. R. |
| 5 | L3 | 18 | ~30 |
| 6 | B5 (Co) | 18 | ~8 |
| 7 | No catalyst | 24 | trace |
| 8 | A4 (Acridinium) | 24 | ~3 |
| 9 | FeCl₂(THF)_{1.5} | 13 | ~28 |
| 10 | FeCl₂(THF)_{1.5}(No LED) | 13 | 0 |
| 11 | FeCl₃ | 13 | ~23 |
| 12 | FeCl₃ (No LED) | 13 | 0 |
| 15 | A5 | 15 | >95 |
| 16 | FeCl₂(THF)_{1.5} + L3 | 15 | ~95 |
| 17 | FeCl₃(H₂O)₆ + L3^{d} | 18 | >95 |
| 18 | FeCl₃(H₂O)₆ + L2^{d} | 18 | ~3 |

^{a}The reaction was ran with 1 (0.2 mmol), 2 (1.0 mmol), 2 (1.0 mmol) and catalyst (0.006 mmol) in MeCN (2 mL).
^{b}NMR yield by using 1,3,5-trimethoxybenzene as internal standard.
^{c}Ran outside at 19° C.
^{d}Weighed all compounds outside, and degassed solvent for 15 mins then the Schlenk tube for 15 mins without any Schlenk technique.

As can be seen, in some instances only a minor trace of product was observed with acridinium complex (e.g., compound of Formula A-I) while use of helicenium compounds of the invention (e.g., compound of Formula A-II) gave a much higher yield. Furthermore, it should be noted that when all compounds were weighted including FeCl₃.(H₂O)₆ and solvent without using a Schlenck flask, and the combined reaction mixture was degassed for 15 mins, the reaction gave a high yield of the product with helicenium ligand L3 as a photocatalyst was used (>95% yield), while only 3% yield of product was observed with acridinium ligand L2. Accordingly, some embodiments of the invention include a method of photocatalyst reaction using compound of Formula A-II optionally without a need for inert atmosphere during combination of reagents.

TABLE B
Catalyst loading

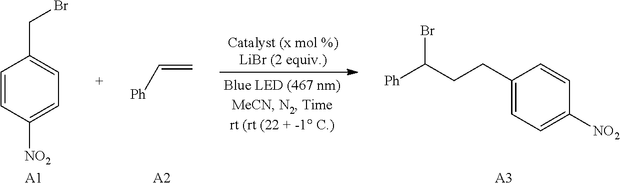

| Entry^{a} | Catalyst | x mol % | Time [h] | Yield [%]^{b} |
|---|---|---|---|---|
| 1 | A5 | 3 | 13 | ~81 |
| 2 | A5 | 3 | 3 | ~18 |
| 3 | A5 | 3 | 6 | ~36 |
| 4 | A5 | 3 | 15 | >95 |
| 5 | A5 | 3 | 20 | >95 |
| 6 | A5 | 1 | 15 | >95 |
| 7 | A5 | 0.5 | 15 | >95 |
| 8 | A5 | 0.2 | 15 | ~67 |
| 9 | A5 | 0.1 | 15 | ~42 |
| 10 | A5 | 0.2 | 20 | ~82 |
| 11 | A5 | 0.1 | 20 | ~55 |
| 12 | A5 | 0.1 | 24 | ~57 |
| 13 | FeCl₂(THF)_{1.5} + L3 | 0.1 | 24 | ~55 |
| 14 | A5 (1.2 equiv. 2) | 0.5 | 15 | ~30 |
| 15 | A5 (2.0 equiv. 2) | 0.5 | 20 | ~50 |

^{a}The reaction was ran with 1 (0.2 mmol), 2 (1.0 mmol), LiBr (0.4 mmol) and catalyst (X mmol) in MeCN (2 mL).
^{b}NMR yield by using 1,3,5-trimethoxybenzene as internal standard.

Table B shows the results of using different amounts of photocatalyst of the invention in addition reaction. Based on these results, the present inventors have discovered that for addition reaction (such as those illustrated in Table A and B), the amount of catalyst of the invention used in methods of the invention is about 5 mol % or less, typically about 3 mol % or less, often about 1 mol % or less, and most often about 0.5 mol % or less.

The reaction temperature can vary depending on a variety of factors, such as but not limited to, reagents used, the amount of reagents used, a particular reaction desired (e.g., addition reaction, radical cyclization reaction, hydrogenation, etc.), reaction solvent, etc. However, in general a photocatalyst reaction using compounds of the invention will range in temperature from about 0° C. to about 140° C. or the boiling point of the solvent, whichever is lower, typically the reaction temperature of a photocatalyst reaction is from about 0° C. to about 100° C., often from about 5° C. to about 80° C., more often from about 10° C. to about 50° C., and most often from about 20° C. to about 30° C.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Unless otherwise specified, all reactions were carried out in oven-dried (overnight) vials or Schlenk tubes with magnetic stirring in a glove box. ¹H, ¹³C and ¹⁹F NMR spectra were recorded on Bruker Avance III-400 MHz or DRX-500 MHz spectrometers in appropriate solvents using TMS as internal standard or the solvent signals as secondary standards. The chemical shifts are shown in 6 scales. Multiplicities of ¹H NMR signals are designated as s (singlet), d (doublet), dd (doublet of doublet), dt (doublet of triplet), t (triplet), quin (quintet), m (multiplet), etc. Compounds were named using ChemDraw and assignments of NMR spectra were done using MestReNova. All chemicals and solvents were purchased from Sigma Aldrich, Fisher Scientific, or VWR. Organic solvents used were dried by a standard solvent purification system (J. C. Meyer or Vigor Solvent Systems). Commercially obtained reagents were used without further purification. All reactions were monitored by TLC silica gel 60 F₂₅₄ (EMD Millipore). Flash column chromatography was carried out using SiliaFlash silica gels F60, 40-63 μm, 60 Å (SiliCycle) at increased pressure. All reactions were performed under N₂ using standard Schlenk techniques or in a glove box (Mbraun glove box).

Synthesis and characterization of dimethoxyquinacridinium (DMQA⁺) tetrafluoroborate 3.

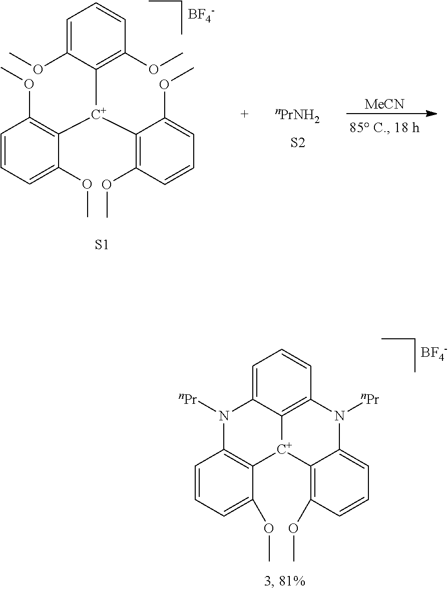

A solution of S1 (1.02 g, 2 mmol, 1.0 equiv.) and S2 (2.95 g, 50 mmol, 25.0 equiv.) in MeCN (20 mL) was stirred at 85° C. for 18 hours. The mixture was cooled to room temperature, transferred to a 250 mL round bottom flask and a large excess of Et₂O (200 mL) was added to crash out the crude product 3 as a dark green solid (924 mg). After layering in DCM/MeCN/MeOH/Et₂O for 2 days, 811 mg of pure product 3 was obtained as a dark green solid after filtration.

Reaction setup for photocatalysis. Two 25 mL Schlenk tubes were placed in a water bath (Pyrex crystallizing dish, 125×65 mm, No. 3140) at the center of a stir plate. Two parallel Red LED lamps (KSPR160L-640-C Red LED 640 nm Photoredox Light customized wavelength) were placed perpendicular to the sidewall of Schlenk tubes, so that the two tubes can be equally exposed to the LEDs. The stir plate/water bath/LEDs were surrounded by an open-top cardboard box covered with aluminum foil to increase the light reflections. A fan (Honeywell, TurboForce Power HT900) over the water bath was turned on when the reaction was running. The combination of an overhead fan and a water bath was to offset the heat generated from the LED lights and stabilize reaction temperature for reproducible results. The water bath was refilled with room temperature deionized water every 12-18 hours. With the above setup, the reaction temperature was maintained at 21-23° C. during the reaction.

General Procedure for Red-Light-Mediated Dual Pd/ⁿPr-DMQA⁺-Catalyzed C—H Arylation.

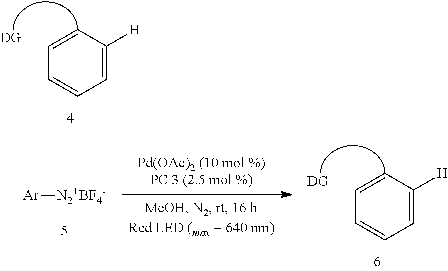

In a N₂ glove box, Pd(OAc)₂ (4.5 mg, 0.02 mmol, 10 mol %), 3 (2.5 mg, 0.005 mmol, 2.5 mol %), the substrate 4 (0.2 mmol, 1.0 equiv.) and the aryldiazonium salt 5 (0.8 mmol, 4.0 equiv.) were added to an oven-dried (overnight) Schlenk tube containing a stirring bar, followed by adding degassed anhydrous MeOH (2.0 mL, 0.1 M). The Schlenk tube was then sealed, removed from the glove box and stirred at room temperature under red LED (λₘₐₓ=640 nm) irradiation. After 16 hours, the mixture was quenched with a saturated solution of NaHCO₃ (2 mL), followed by adding deionized water (2 mL). The crude reaction mixture was then extracted with ethyl acetate, and the combined organic layers were washed with brine and dried over anhydrous Na₂SO₄. After filtration, the solvent was removed under reduced pressure. The crude product was purified by flash chromatography (FC) on silica gel (eluent: Hexanes/EtOAc=20/1˜1/1) to yield the desired product 6.

1-([1,1′-biphenyl]-2-yl)pyrrolidin-2-one (6aa): Yield (44 mg, 93%). A clear viscous oil. R_{f}=0.2 (Hexanes/EtOAc=1/2). FC (Hexanes/EtOAc=1/1). ¹H NMR (400 MHz, C₆D₆) δ 7.39-7.35 (m, 2H, ArH), 7.33 (dd, J=8.0, 1.6 Hz, 1H, ArH), 7.21 (dd, J=7.6, 1.6 Hz, 1H, ArH), 7.17-7.04 (m, 5H, ArH), 2.78 (t, J=6.8 Hz, 2H, CH₂), 2.04 (t, J=8.0 Hz, 2H, CH₂), 1.19 (tt, J=6.8, 8.0 Hz, 2H, CH₂). ¹³C NMR (101 MHz, C₆D₆) δ 174.18, 140.13, 140.08, 137.67, 130.98, 129.18, 128.82, 128.63, 128.48, 127.74, 127.64, 49.70, 31.11, 19.06. ¹H NMR (400 MHz, CDCl₃) δ 7.43-7.31 (m, 9H, ArH), 3.21 (t, J=6.8 Hz, 2H, CH₂), 2.42 (t, J=8.0 Hz, 2H, CH₂), 1.87 (tt, J=6.8, 8.0 Hz, 2H, CH₂). ¹³C NMR (101 MHz, CDCl₃) δ 175.70, 139.73, 139.22, 136.43, 130.92, 128.65, 128.51, 128.49, 128.45, 128.12, 127.67, 50.26, 31.30, 19.08.

1-(4′-chloro-[1,1′-biphenyl]-2-yl)pyrrolidin-2-one (6ab): Yield (50 mg, 92%). A light yellow solid. R_{f}=0.2 (Hexanes/EtOAc=1/2). FC (Hexanes/EtOAc=1/1). ¹H NMR (400 MHz, C₆D₆) δ 7.24 (dd, J=8.0, 1.2 Hz, 1H, ArH), 7.15-7.04 (m, 7H, ArH), 2.73 (t, J=6.8 Hz, 2H, CH₂), 2.00 (t, J=8.0 Hz, 2H, CH₂), 1.18 (tt, J=6.8, 8.0 Hz, 2H, CH₂). ¹³C NMR (101 MHz, C₆D₆) δ 174.18, 138.86, 138.46, 137.53, 133.78, 130.79, 130.21, 129.01, 128.82, 128.81, 49.77, 31.03, 19.01. ¹H NMR (400 MHz, CDCl₃) δ 7.44-7.30 (m, 8H, ArH), 3.26 (t, J=6.8 Hz, 2H, CH₂), 2.43 (t, J=8.0 Hz, 2H, CH₂), 1.92 (tt, J=6.8, 8.0 Hz, 2H, CH₂). ¹³C NMR (126 MHz, CDCl₃) δ 175.83, 138.71, 137.78, 136.49, 133.87, 130.85, 129.88, 129.10, 128.79, 128.58, 128.34, 128.33, 50.33, 31.13, 18.95.

1-(4′-methoxy-[1,1′-biphenyl]-2-yl)pyrrolidin-2-one (6ac): Yield (46 mg, 86%). An orange viscous oil. R_{f}=0.2 (Hexanes/EtOAc=1/2). FC (Hexanes/EtOAc=1/1).

¹H NMR (400 MHz, CDCl₃) δ 7.38-7.34 (m, 3H, ArH), 7.32-7.28 (m, 3H, ArH), 6.94-6.92 (m, 2H, ArH), 3.84 (s, 3H), 3.22 (t, J=6.8 Hz, 2H, CH₂), 2.44 (t, J=8.0 Hz, 2H, CH₂), 1.89 (tt, J=6.8, 8.0 Hz, 2H, CH₂). ¹³C NMR (101 MHz, CDCl₃) δ 175.76, 159.25, 139.37, 136.42, 131.58, 130.94, 129.59, 128.49, 128.30, 128.12, 113.96, 55.37, 50.18, 31.35, 19.11.

2-(3-methyl-[1,1′-biphenyl]-2-yl)pyridine (6ba): Yield (36 mg, 73%). A clear viscous oil. R_{f}=0.4 (Hexanes/EtOAc=5/1). FC (Hexanes/EtOAc=20/1). ¹H NMR (500 MHz, CDCl₃) δ 8.63 (ddd, J=5.0, 2.0, 1.0 Hz, 1H, ArH), 7.44 (ddd, J=7.5, 7.5, 1.5 Hz, 1H, ArH), 7.36 (dd, J=7.5, 7.5 Hz, 1H, ArH), 7.31-7.26 (m, 2H, ArH), 7.16-7.11 (m, 3H, ArH), 7.10-7.06 (m, 3H, ArH), 6.88 (ddd, J=7.5, 7.5, 1.5 Hz, 1H, ArH), 2.19 (s, 3H, Me). ¹³C NMR (101 MHz, CDCl₃) δ 159.69, 148.92, 141.77, 141.36, 139.41, 136.80, 135.81, 129.75, 129.71, 129.52, 128.15, 127.70, 127.69, 126.32, 125.74, 121.40, 20.59.

2-(4′-chloro-3-methyl-[1,1′-biphenyl]-2-yl)pyridine (6bb): Yield (38 mg, 68%). A pale yellow viscous oil. R_{f}=0.4 (Hexanes/EtOAc=5/1). FC (Hexanes/EtOAc=20/1). ¹H NMR (400 MHz, CDCl₃) δ 8.62 (ddd, J=4.8, 4.8, 1.2 Hz, 1H, ArH), 7.48 (ddd, J=7.6, 7.6, 2.0 Hz, 1H, ArH), 7.38-7.28 (m, 2H, ArH), 7.23 (dd, J=7.6, 1.2 Hz, 1H, ArH), 7.13-7.09 (m, 3H, ArH), 7.03-6.99 (m, 2H, ArH), 6.89 (ddd, J=7.6, 7.6, 1.2 Hz, 1H, ArH), 2.18 (s, 3H, Me). ¹³C NMR (101 MHz, CDCl₃) δ 159.40, 149.11, 140.26, 140.07, 139.42, 136.97, 136.00, 132.46, 130.99, 129.83, 128.24, 127.91, 127.53, 125.64, 121.58, 20.55.

2-(4′-methoxy-3-methyl-[1,1′-biphenyl]-2-yl)pyridine (6bc): Yield (33 mg, 60%). A clear viscous oil. R_{f}=0.4 (Hexanes/EtOAc=5/1). FC (Hexanes/EtOAc=20/1). ¹H NMR (400 MHz, CDCl₃) δ 8.63 (ddd, J=4.8, 4.8, 1.2 Hz, 1H, ArH), 7.46 (ddd, J=7.6, 7.6, 2.0 Hz, 1H, ArH), 7.34 (dd, J=7.6, 7.6 Hz, 1H, ArH), 7.28-7.23 (m, 2H, ArH), 7.09 (ddd, J=7.6, 4.8, 1.2 Hz, 1H, ArH), 7.02-6.97 (m, 2H, ArH), 6.88 (d, J=8.0 Hz, 1H, ArH), 6.70-6.66 (m, 2H, ArH), 3.73 (s, 3H, OMe), 2.18 (s, 3H, Me). ¹³C NMR (101 MHz, CDCl₃) δ 159.93, 158.17, 148.98, 140.90, 139.45, 136.78, 135.86, 134.22, 130.78, 129.18, 128.11, 127.69, 125.71, 121.33, 113.18, 55.20, 20.60.

2-(4-methyl-[1,1′-biphenyl]-2-yl)pyrimidine (6ca): Yield (28 mg, 57%). A clear viscous oil. R_{f}=0.3 (Hexanes/EtOAc=3/1). FC (Hexanes/EtOAc=15/1). ¹H NMR (400 MHz, CDCl₃) δ 8.63 (d, J=4.8 Hz, 2H, ArH), 7.61 (d, J=1.6 Hz, 1H, ArH), 7.37 (d, J=7.6 Hz, 1H, ArH), 7.32 (dd, J=7.6, 1.6 Hz, 1H, ArH), 7.24-7.18 (m, 3H, ArH), 7.14-7.10 (m, 2H, ArH), 7.08 (dd, J=4.8, 4.8 Hz, 1H, ArH), 2.46 (s, 3H, Me). ¹³C NMR (101 MHz, CDCl₃) δ 168.37, 156.83, 141.67, 138.76, 138.12, 137.28, 131.19, 130.79, 130.29, 129.28, 128.05, 126.39, 118.50, 21.18.

1-(4-methyl-[1,1′-biphenyl]-2-yl)ethan-1-one oxime (6da): Yield (29 mg, 64%). A white solid. R_{f}=0.4 (Hexanes/EtOAc=5/1). FC (Hexanes/EtOAc=20/1). ¹H NMR (400 MHz, C₆D₆) δ 8.69 (s, 1H, OH), 7.40-7.37 (m, 2H, ArH), 7.32 (d, J=2.0 Hz, 1H, ArH), 7.13-7.03 (m, 3H, ArH), 6.96 (dd, J=7.6, 2.0 Hz, 1H, ArH), 2.09 (s, 3H, Me), 1.78 (s, 3H, Me). ¹³C NMR (101 MHz, C₆D₆) δ 159.07, 141.64, 138.32, 137.39, 137.27, 130.55, 130.44, 129.86, 129.37, 128.69, 127.31, 20.85, 16.30. ¹H NMR (500 MHz, CDCl₃) δ 8.15 (s, 1H, OH), 7.42-7.37 (m, 4H, ArH), 7.35-7.31 (m, 1H, ArH), 7.30-7.28 (m, 1H, ArH), 7.27-7.24 (m, 2H, ArH), 2.41 (s, 3H, Me), 1.69 (s, 3H, Me). ¹³C NMR (101 MHz, CDCl₃) δ 159.50, 141.10, 137.89, 137.31, 136.73, 130.38, 129.90, 129.84, 129.09, 128.54, 127.25, 21.12, 16.12.

Red-light-mediated dual Pd/ⁿPr-DMQA⁺-catalyzed C—H arylation. Using 1-([1,1′-biphenyl]-2-yl)pyrrolidin-2-one (4a) and benzenediazonium tetrafluoroborate (5a) as the model substrates, Pd(OAc)₂ as the catalyst, 3 as the PC, MeOH as the solvent, the reaction time was screened and the results are outlined in Table S2. Under red LED (λₘₐₓ=640 nm), the reaction proceeded smoothly to afford the desired product 6aa in 95% NMR yield after 8 hours (Table S2, entry 1). By decreasing the reaction time to 6 h, 4 h and 2 h, the desired product 6aa was obtained in 95%, 95% and 85% NMR yield, respectively. To test the background reactions, several control experiments have also been performed. In the absence of PC 3, red light or Pd(OAc)₂, significantly lower yields (≤25%) of 6aa was observed for all the conditions.

NMR results for control reactions. To further demonstrate the essential roles of red light, PC 3 and Pd catalyst, control experiments to determine the background reaction yields for all substrates have been conducted and the NMR results were determined. In general, in the absence of red light, ≤15% NMR yield was observed for all the substrates, except for 36% NMR yield for the reaction of 4a and 4-chlorobenzenediazonium tetrafluoroborate 5b. In the absence of 3, ≤40% NMR yield was observed for all the substrates, except for 45% NMR yield for the reaction of 4a and 5b. The lower background observed in the present system highlights the great potential of low-energy red light towards reaction selectivity. At last, in the absence of Pd catalyst, trace amount of products 6 was observed for all the substrates, which supports the essential role of palladium for activating the substrate 4 during the catalytic cycle.

General procedure for ⁿPr-DMQA⁺-catalyzed aerobic oxidative hydroxylation of arylboronic acids under red light. To a mixture of arylboronic acid 7 (0.50 mmol, 1.0 equiv.) and PC 3 (5.0 mg, 0.01 mmol, 2 mol %) in DMF (5.0 mL, 0.1 M) was added DIPEA (129 mg, 1.0 mmol, 2.0 equiv.) in a Schlenk tube. The solution was stirred at room temperature under red LED (λₘₐₓ=640 nm) irradiation in open to air (without bubbling air). After 24 hours, the reaction mixture was cooled to 0° C. and quenched by adding aqueous solution of HCl slowly, followed by extracting with Et₂O. The combined organic layers were washed with brine and dried over anhydrous Na₂SO₄. After filtration, the solvent was removed under reduced pressure. The crude product was purified by flash chromatography on silica gel (eluent: Hexanes/EtOAc=20/1˜1/2) to furnish the desired product 8.

Phenol (8a): Yield (39 mg, 83%). A colorless solid. R_{f}=0.4 (Hexanes/EtOAc=5/1). FC (Hexanes/EtOAc=15/1). ¹H NMR (500 MHz, CDCl₃) δ 7.27-7.23 (m, 2H, ArH), 6.96-6.92 (m, 1H, ArH), 6.87-6.83 (m, 2H, ArH), 5.10 (s, 1H, OH). ¹³C NMR (126 MHz, CDCl₃) δ 155.75, 129.89, 120.97, 115.50.

4-Methoxyphenol (8b): Yield (44 mg, 71%). A colorless solid. R_{f}=0.2 (Hexanes/EtOAc=5/1). FC (Hexanes/EtOAc=10/1). ¹H NMR (400 MHz, CDCl₃) δ 6.84-6.77 (m, 4H, ArH), 4.76 (s, 1H, OH), 3.80 (s, 3H, OMe). ¹³C NMR (101 MHz, CDCl₃) δ 153.89, 149.59, 116.19, 115.02, 55.96.

4-Chlorophenol (8c): Yield (52 mg, 81%). A colorless oil. R_{f}=0.4 (Hexanes/EtOAc=5/1). FC (Hexanes/EtOAc=10/1). ¹H NMR (400 MHz, CDCl₃) δ 7.21-7.18 (m, 2H, ArH), 6.78-6.75 (m, 2H, ArH), 4.86 (s, 1H, OH). ¹³C NMR (101 MHz, CDCl₃) δ 154.22, 129.67, 125.83, 116.80.

4-Cyanophenol (8d): Yield (52 mg, 87%). A light yellow solid. R_{f}=0.1 (Hexanes/EtOAc=3/1). FC (Hexanes/EtOAc=5/1). ¹H NMR (400 MHz, CDCl₃) δ 7.57-7.54 (m, 2H, ArH), 6.96-6.93 (m, 2H, ArH), 6.69 (s, 1H, OH). ¹³C NMR (101 MHz, CDCl₃) δ 160.34, 134.47, 119.37, 116.61, 103.16.

4-Hydroxybenzaldehyde (8e): Yield (40 mg, 66%). A white solid. R_{f}=0.3 (Hexanes/EtOAc=3/1). FC (Hexanes/EtOAc=5/1). ¹H NMR (400 MHz, Methanol-d₄) δ 9.76 (s, 1H, CHO), 7.78-7.75 (m, 2H, ArH), 6.93-6.90 (m, 2H, ArH). ¹³C NMR (101 MHz, Methanol-d₄) δ 192.81, 165.15, 133.42, 130.30, 116.85.

Methyl 4-hydroxybenzoate (8f): Yield (61 mg, 80%). A white solid. R_{f}=0.3 (Hexanes/EtOAc=3/1). FC (Hexanes/EtOAc=5/1). ¹H NMR (400 MHz, CDCl₃) δ 7.97-7.93 (m, 2H, ArH), 6.91-6.87 (m, 2H, ArH), 6.46 (s, 1H, OH), 3.90 (s, 3H, OMe). ¹³C NMR (101 MHz, CDCl₃) δ 167.65, 160.44, 132.11, 122.39, 115.45, 52.26.

4-Hydroxybenzoic acid (8g): Yield (28 mg, 41%). A white solid. R_{f}=0.2 (Hexanes/EtOAc=1/1). FC (Hexanes/EtOAc=1/1). ¹H NMR (400 MHz, Methanol-d₄) δ 7.89-7.86 (m, 2H, ArH), 6.83-6.80 (m, 2H, ArH). ¹³C NMR (126 MHz, Methanol-d₄) δ 170.26, 163.54, 133.13, 122.81, 116.13.

o-Cresol (8h): Yield (39 mg, 72%). A light yellow oil. R_{f}=0.5 (Hexanes/EtOAc=5/1). FC (Hexanes/EtOAc=20/1). ¹H NMR (500 MHz, CDCl₃) δ 7.14-7.11 (m, 1H, ArH), 7.11-7.06 (m, 1H, ArH), 7.14-7.11 (m, 1H, ArH), 6.77 (dd, J=8.0, 1.5 Hz, 1H, ArH), 4.65 (s, 1H, OH), 2.26 (s, 3H, Me). ¹³C NMR (101 MHz, CDCl₃) δ 153.89, 131.16, 127.27, 123.80, 120.89, 115.01, 15.84.

2-Methoxyphenol (8i): Yield (34 mg, 55%). A white solid. R_{f}=0.5 (Hexanes/EtOAc=5/1). FC (Hexanes/EtOAc=20/1). ¹H NMR (500 MHz, CDCl₃) δ 6.94-6.91 (m, 1H, ArH), 6.90-6.84 (m, 3H, ArH), 5.60 (s, 1H, OH), 3.89 (s, 3H, OMe). ¹³C NMR (126 MHz, CDCl₃) δ 146.82, 145.92, 121.66, 120.34, 114.72, 110.90, 55.97.

m-Cresol (8j): Yield (36 mg, 67%). A colorless oil. R_{f}=0.5 (Hexanes/EtOAc=5/1). FC (Hexanes/EtOAc=20/1). ¹H NMR (400 MHz, CDCl₃) δ 7.13 (dd, J=7.6, 8.0 Hz, 1H, ArH), 6.76 (d, J=7.6 Hz, 1H, ArH), 6.68-6.62 (m, 2H, ArH), 4.76 (s, 1H, OH), 2.32 (s, 3H, Me). ¹³C NMR (101 MHz, CDCl₃) δ 155.53, 139.97, 129.56, 121.77, 116.15, 112.40, 21.48.

3-Nitrophenol (8k): Yield (51 mg, 73%). A white solid. R_{f}=0.4 (Hexanes/EtOAc=3/1). FC (Hexanes/EtOAc=10/1). ¹H NMR (400 MHz, CDCl₃) δ 7.81 (dd, J=8.4, 2.0 Hz, 1H, ArH), 7.71 (dd, J=2.4, 2.0 Hz, 1H, ArH), 7.41 (dd, J=8.4, 8.4 Hz, 1H, ArH), 7.19 (dd, J=8.4, 2.4 Hz, 1H, ArH), 5.63 (s, 1H, OH). ¹³C NMR (101 MHz, CDCl₃) δ 156.39, 149.25, 130.46, 122.16, 116.08, 110.70.

Naphthalen-2-ol (8l): Yield (47 mg, 65%). A light yellow solid. R_{f}=0.3 (Hexanes/EtOAc=5/1). FC (Hexanes/EtOAc=15/1). ¹H NMR (400 MHz, CDCl₃) δ 7.78 (dd, J=8.4, 8.4 Hz, 2H, ArH), 7.69 (d, J=8.4 Hz, 1H, ArH), 7.48-7.43 (m, 1H, ArH), 7.38-7.33 (m, 1H, ArH), 7.16 (d, J=2.4 Hz, 1H, ArH), 7.12 (dd, J=8.8, 2.4 Hz, 1H, ArH), 5.16 (s, 1H, OH). ¹³C NMR (101 MHz, CDCl₃) δ 153.36, 134.70, 130.01, 129.09, 127.90, 126.68, 126.51, 123.79, 117.84, 109.67.

Optimization of ⁿPr-DMQA⁺-catalyzed aerobic oxidative hydroxylation. Using phenylboronic acid 7a as the model substrate, DIPEA as the base, 3 as the PC, air as the oxidant, DMF as the solvent, the reaction time was screened. Under red LED (λₘₐₓ=640 nm), the reaction proceeded smoothly to afford phenol 8a in 77% NMR yield after 18 hours. By increasing the reaction time to 24 hours, 8a was obtained in 87% NMR yield, along with 83% isolated yield. Running the reaction for 28 h did not improve the result. Several control experiments have also been performed. In the absence of 3 or red light, little or no conversion was observed.

Red-light-induced ⁿPr-DMQA⁺-catalyzed oxygenation. To a mixture of tertiary amine 9 (42 mg, 0.20 mmol, 1.0 equiv.) and PC 3 (2.5 mg, 0.005 mmol, 2.5 mol %) in DMF (2.0 mL, 0.1 M) was added DBU (46 mg, 0.3 mmol, 1.5 equiv.) in a Schlenk tube. The solution was stirred at room temperature under red LED (λₘₐₓ=640 nm) irradiation in open to air (without bubbling air) for 20 hours. The mixture was concentrated in vacuo to yield the crude product, which was purified by flash chromatography on silica gel (eluent: Hexanes/EtOAc=10/1˜5/1) to yield the desired amide 10 as a white solid.

2-phenyl-3,4-dihydroisoquinolin-1(2H)-one (10): Yield (41 mg, 92%). A white solid. R_{f}=0.3 (Hexanes/EtOAc=3/1). FC (Hexanes/EtOAc=10/1˜5/1). ¹H NMR (400 MHz, CDCl₃) δ 8.17 (d, J=7.6 Hz, 1H, ArH), 7.55-7.36 (m, 6H, ArH), 7.29-7.24 (m, 2H, ArH), 4.01 (t, J=6.4 Hz, 2H, CH₂), 3.16 (t, J=6.4 Hz, 2H, CH₂). ¹³C NMR (101 MHz, CDCl₃) δ 164.29, 143.24, 138.42, 132.13, 129.85, 129.02, 128.86, 127.30, 127.05, 126.34, 125.43, 49.53, 28.75.

Red-light-induced ⁿPr-DMQA⁺-catalyzed ATRA. In a N₂ glove box, 3 (1.0 mg, 0.002 mmol, 1 mol %), 4-nitrobenzyl bromide 11 (43 mg, 0.2 mmol, 1.0 equiv.) and LiBr (35 mg, 0.4 mmol, 2.0 equiv.) were added to an oven-dried (overnight) Schlenk tube containing a stirring bar, followed by adding dry MeCN (1.0 mL, 0.2 M) and styrene 12 (208 mg, 2.0 mmol, 10.0 equiv.). The Schlenk tube was then sealed, removed from the glove box and stirred at room temperature under red LED (λₘₐₓ=640 nm) irradiation for 17 hours. The mixture was concentrated in vacuo to yield the crude product, which was purified by flash chromatography (FC) on silica gel (eluent: Hexanes/EtOAc=200/1) to yield the desired product 13 as a colorless oil.

1-(3-bromo-3-phenylpropyl)-4-nitrobenzene (13): Yield (38 mg, 59%). A colorless oil. R_{f}=0.4 (Hexanes/EtOAc=10/1). FC (Hexanes/EtOAc=200/1). ¹H NMR (400 MHz, CDCl₃) δ 8.18-8.14 (m, 2H, ArH), 7.40-7.28 (m, 7H, ArH), 4.88 (dd, J=8.4, 6.0 Hz, 1H, CH), 2.95 (ddd, J=14.4, 9.2, 5.6 Hz, 1H, CH₂), 2.82 (ddd, J=14.4, 8.8, 6.4 Hz, 1H, CH₂), 2.69-2.59 (m, 1H, CH₂), 2.50-2.41 (m, 1H, CH₂). ¹³C NMR (101 MHz, CDCl₃) δ 148.37, 146.77, 141.53, 129.47, 128.99, 128.78, 127.34, 123.94, 54.09, 40.90, 34.30.

Red-light-induced ⁿPr-DMQA⁺-catalyzed intermolecular ATRA. Initial examination of ATRA reaction was tested by reacting 4-nitrobenzyl bromide 11 (1.0 equiv.) with styrene 12 (5.0 equiv.) in the presence of LiBr (2.0 equiv.) and PC 3 in MeCN under red LED (λₘₐₓ=640 nm). Running the reaction with 1 mol % of 3 at rt for 20 hours delivered the desired adduct 13 in 40% NMR yield, along with 50% starting material 11 (“SM-11”). Increasing the PC loading of 3 to 2.5 mol % gave lower yield of 13, along with more byproducts, which could be the dimer, polymers or other side-products from the radical intermediates. Thus, 1 mol % the PC loading was chosen for all the follow-up screenings. When the reaction was run at 50° C. for 24 hours, only 25% of desired product 13 was observed, along with a lot of byproducts and no SM-11. Two control experiments have also been performed, and no reaction occurred at 50° C. in the absence of red light or PC 3. Performing the reaction at 35° C. didn't improve any reaction result. When the reaction was conducted in a higher concentration (0.2 M), the reaction yield was improved from 40% to 54%. Moreover, when the reaction was performed with 10.0 equiv. of styrene 12 in a higher concentration (0.2 M), 13 was achieved in 65% yield, along with 12% SM-11. Further increasing the reaction concentration (0.4 M) did not increase the reaction yield.

Based on the above results, further screening the solvents and reaction times were carried out. The examination of solvent effects revealed that the reaction in MeCN provided a higher yield than those in DMF/H₂O (1/4), MeOH and DMSO. Increasing the reaction time from 17 to 24 hours in MeCN gave the product 13 in 59% yield along with trace amount of SM-11. Several control experiments have also been conducted. No reaction occurred in the absence of 3 or red light, which is consistent with the results at 50° C. In the absence of LiBr, 13 was obtained in 14% yield along with 75% of SM-11.

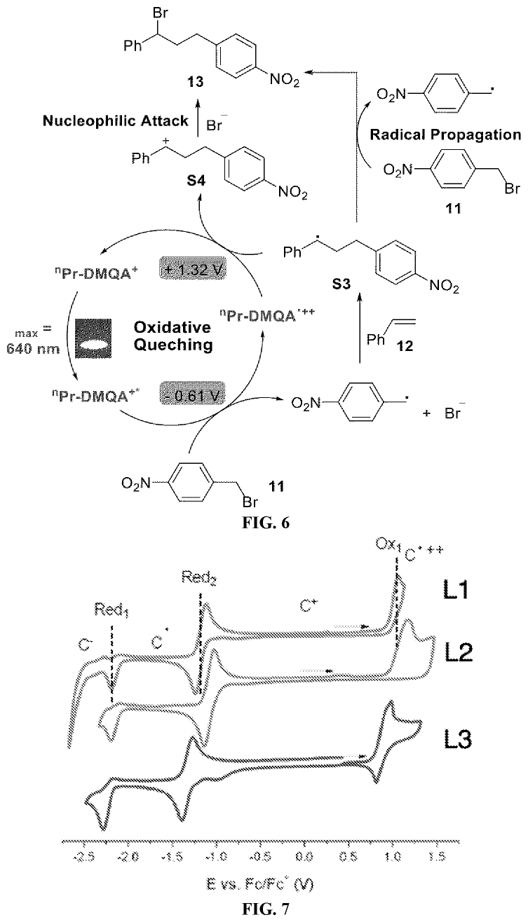

Mechanism for red-light-induced ⁿPr-DMQA⁺-catalyzed intermolecular ATRA. Without being bound by any theory, one possible reaction mechanism for the red-light-induced ⁿPr-DMQA⁺-catalyzed intermolecular ATRA illustrated in FIG. 6. Firstly, oxidative quenching of the red light-induced excited state of ⁿPr-DMQA⁺*, by 4-nitrobenzyl bromide (11) generates a 4-nitrobenzyl radical, along with bromide anion. Then, addition of the 4-nitrobenzyl radical to styrene (12) forms another benzyl radical intermediate S3, which undergoes a single electron transfer (SET) process with ⁿPr-DMQA.⁺⁺ to regenerate the PC ⁿPr-DMQA⁺ and form the carbocation species S4 (PhCH₂⁺/PhCH₂.=+0.73V vs SCE). Final product 13 is formed by the nucleophilic attack of bromide anion to carbocation species S4. Alternatively, 13 could be also obtained through a radical chain transfer mechanism. 14.

Red-light-mediated dual Au/ⁿPr-DMQA⁺-catalyzed C(sp)-H arylation. In a N₂ glove box, Au(PPh₃)Cl (9.9 mg, 0.02 mmol, 10 mol %), 3 (2.5 mg, 0.005 mmol, 2.5 mol %), 1-ethynyl-4-methylbenzene 14 (24 mg, 0.2 mmol, 1.0 equiv.) and benzenediazonium tetrafluoroborate 5a (154 mg, 0.8 mmol, 4.0 equiv.) were added to an oven-dried (overnight) Schlenk tube containing a stirring bar, followed by adding dry DMF (2.0 mL, 0.1 M). The Schlenk tube was then sealed, removed from the glove box and stirred at room temperature under red LED (λₘₐₓ=640 nm) irradiation. After 1 hour, the mixture was quenched with a saturated solution of NaHCO₃, followed by adding deionized water. The crude reaction mixture was then extracted with ethyl acetate, and the combined organic layers were washed with brine and dried over anhydrous Na₂SO₄. After filtration, the solvent was removed under reduced pressure. The crude product was purified by flash chromatography on silica gel (eluent: Hexanes/EtOAc=Hexanes ˜200/1) to yield the desired product 15 as a white solid.

1-methyl-4-(phenylethynyl)benzene (15): Yield (24 mg, 62%). A white solid. R_{f}=0.45 (Hexanes). FC (Hexanes/EtOAc=Hexanes ˜200/1). ¹H NMR (400 MHz, CDCl₃) δ 7.56-7.53 (m, 2H, ArH), 7.45 (d, J=7.6 Hz, 2H, ArH), 7.38-7.31 (m, 3H, ArH), 7.17 (d, J=7.6 Hz, 2H, ArH), 2.39 (s, 3H). ¹³C NMR (101 MHz, CDCl₃) δ 138.51, 131.68, 131.63, 129.25, 128.45, 128.20, 123.63, 120.33, 89.70, 88.86, 21.65.

Red-light-mediated dual Au/ⁿPr-DMQA⁺-catalyzed C(sp)-H arylation. Optimization of C(sp)-H arylation by screening the solvents were performed. The examination of solvent effects showed that the reaction in DMF gave a higher yield than those in DMSO and MeOH. Control experiments have also been conducted. In the absence of 3 or red light, and the desired product 15 was obtained in 24% and 5% yield, respectively, while the reaction was messy in the absence of Au(PPh₃)Cl.

NMR results for control reactions for Scheme 2. Several control experiments have also been performed for the red-light-induced aerobic benzylic C(sp³)-H oxygenation, intermolecular ATRA and dual Au/ⁿPr-DMQA⁺-catalyzed C(sp)-H arylation (Scheme 2). For aerobic benzylic C(sp³)-H oxygenation, no conversion was observed in the absence of PC 3 or red light, while the reaction was messy in the absence of DBU. For intermolecular ATRA, no reaction occurred in the absence of PC 3 or red light, while the desired adduct 13 was obtained in 14% yield along with 75% of SM-11 in the absence of LiBr. For dual Au/ⁿPr-DMQA⁺-catalyzed C(sp)-H arylation, in the absence of PC 3 or red light, the desired product 15 was formed in 24% and 5% yield, respectively, while the reaction was messy in the absence of Au(PPh₃)Cl.

General Procedure of the Preparation of the Carbocation-Based Pyridine-Containing Ligands L1-L3.

2-(2-Aminoethyl)pyridine (0.58 mL, 4.8 mmol, 1.2 equiv.) was added to a suspension of 1 (2.04 g, 4.0 mmol, 1.0 equiv.) in ethyl acetate (100 mL). The solution was stirred at rt for 2 hours. Then 150 mL ethyl ether was added and stirred at rt for 30 mins. The red solid was filtered and further purified by recrystallization in DCM/hexane to yield large dark red crystals (2.04 g, 90%).

L1: Yield: (2.04 g, 90%). A dark red solid. M.P.: 220-222° C. ¹H NMR (400 MHz, CDCl₃) δ 8.58 (ddd, J=4.8, 2.0, 0.8 Hz, 1H, Py), 8.26 (dd, J=9.2, 8.0 Hz, 2H, ArH), 8.19 (d, J=9.2 Hz, 2H, ArH), 7.70 (ddd, J=7.6, 7.6, 2.0 Hz, 1H, Py), 7.57 (ddd, J=7.6, 0.8, 0.8 Hz, 1H, Py), 7.37 (dd, J=8.4, 8.4 Hz, 1H, ArH), 7.21 (ddd, J=7.6, 4.8, 0.8 Hz, 1H, Py), 7.02 (d, J=8.0 Hz, 2H, ArH), 6.66 (d, J=8.4 Hz, 2H, ArH), 5.53 (t, J=8.0 Hz, 2H, NCH₂CH₂Py), 3.69 (t, J=8.0 Hz, 2H, NCH₂CH₂Py), 3.56 (s, 6H, OMe), 3.58 (s, 6H, OMe). ¹³C NMR (101 MHz, CDCl₃) δ 160.90, 157.83, 156.23, 149.55, 141.67, 140.84, 137.53, 129.62, 124.60, 122.66, 119.91, 119.50, 109.15, 106.62, 103.54, 57.04, 56.08, 52.03, 35.66. ¹⁹F NMR (376 MHz, CDCl₃) δ −153.42, −153.48. HRMS (ESI) Calcd. for C30H29N2O4+1(M⁺+1) requires 481.2122. Found: 481.2119.

2-(2-Aminomethyl)pyridine (0.13 mL, 1.2 mmol, 1.2 equiv.) was added to a suspension of 1 (0.51 g, 1.0 mmol, 1.0 equiv.) in MeCN (15 mL). The solution was stirred at rt for 1 hours. MeCN was reduced to 5 mL on RotaVap. Then 100 mL Et₂O was added and stirred at rt for 2 hs. The red solid was filtered and further purified by recrystallization in DCM/Et₂O to yield large dark red crystals (419 mg, 76%).

L2: Yield: (419 mg, 76%). A dark red solid. M.P.: 280-282° C. ¹H NMR (500 MHz, DMSO-d₆) δ 8.34 (ddd, J=5.0, 2.0, 1.0 Hz, 1H, Py), 8.17 (dd, J=9.0, 8.0 Hz, 2H, ArH), 7.99 (Py, J=8.0, 8.0 2.0 Hz, 1H, ArH), 7.89 (d, J=9.0 Hz, 2H, ArH), 7.81 (Py, J=8.0, 1.0, 1.0 Hz, 1H, ArH), 7.46 (dd, J=8.5 Hz, 1H, ArH), 7.38 (Py, J=8.0, 5.0, 1.0 Hz, 1H, ArH), 7.20 (d, J=8.0 Hz, 2H, ArH), 6.84 (d, J=8.5 Hz, 2H, ArH), 6.70 (s, 2H, CH₂), 3.57 (s, 6H, OMe), 3.54 (s, 6H, OMe). ¹³C NMR (126 MHz, DMSO-d₆) δ 160.06, 157.06, 153.49, 149.53, 142.26, 140.22, 137.73, 129.53, 123.54, 122.43, 119.35, 119.13, 110.36, 106.97, 103.83, 57.21, 56.16, 55.89. ¹⁹F NMR (376 MHz, DMSO-d₆) δ −148.25, −148.31. HRMS (ESI) Calcd. for C29H27N2O4⁺¹(M⁺+1) requires 467.1965. Found: 467.1965.

A solution of L1 (1.14 g, 2.0 mmol, 1.0 equiv.) and n-propylamine (1.49 mL, 20.0 mmol, 10.0 equiv.) in 20 mL DMF in a pressure flask was stirred at 70° C. for 2 days. A dark green solution was formed. After cooling to rt, DMF was reduced to 4 mL on iRotaVap. 20 mL MeCN was added, followed by adding a large excess of Et₂O (200 mL) and stirring vigorously at rt for 1 h. A lot of dark green solids crashed out, filtered to yield crude product, which was further purified by recrystallization in DCM/Et₂O to yield dark green crystals (742 mg, 66%).

L3: Yield: (742 mg, 66%). A dark green solid. M.P.: 214-216° C. ¹H NMR (400 MHz, DMSO-d₆) δ 8.61-8.58 (m, 1H, Py), 8.24 (dd, J=8.4, 8.4 Hz, 1H, ArH), 7.98-7.92 (m, 2H, ArH), 7.82 (d, J=8.4 Hz, 1H, ArH), 7.77 (ddd, J=7.6, 7.6, 2.0 Hz, 1H, Py), 7.71 (d, J=8.4 Hz, 1H, ArH), 7.68 (d, J=8.4 Hz, 1H, ArH), 7.62 (d, J=8.4 Hz, 1H, ArH), 7.50-7.47 (m, 1H, Py), 7.33-7.29 (m, 1H, Py), 7.03-7.00 (m, 1H, ArH), 5.17-5.08 (m, 1H, CH₂), 4.91-4.83 (m, 1H, CH₂), 4.77-4.68 (m, 1H, CH₂), 4.51-4.42 (m, 1H, CH₂), 3.73 (s, 6H, OMe), 3.44-3.39 (m, 2H, CH₂), 2.05-1.90 (m, 2H, CH₂), 1.17 (t, J=7.2 Hz, 3H, CH₃). ¹³C NMR (126 MHz, DMSO-d₆) δ 159.62, 157.85, 149.90, 142.31, 142.12, 142.02, 138.64, 137.71, 137.68, 137.37, 137.20, 124.44, 122.67, 118.98, 112.74, 108.11, 107.92, 105.61, 105.34, 103.54, 103.47, 56.02, 56.01, 50.91, 49.32, 33.93, 19.71, 11.07. ¹⁹F NMR (376 MHz, DMSO-d₆) δ −148.25, −148.31. HRMS (ESI) Calcd. for C31H30N3O2⁺¹(M⁺+1) requires 476.2333. Found: 476.2331.

Exemplary Procedure of the Synthesis of Co(II) and Ni (II) Complexes 2-4

A solution of L (0.2 mmol, 1.0 equiv.), CoCl₂(THF)_{1.5} or NiCl₂(glyme) (0.2 mmol, 1.0 equiv.) and LiCl (0.4 mmol, 2.0 equiv.) in MeCN (8 mL) was stirred in the glove box at rt for 20 hours. A cloudy solution was formed. The solid was filtered, washed with MeCN, THE and Et₂O, and dried under vacuum to give the final complex 2-4.

2a Co(L1)Cl₃: Yield: (108 mg, 84%). A dark red solid. M.P.: 262-264° C. Elementary Analysis (0.886 mg) requires: C, 55.70; H, 4.52; Cl, 16.44; Co, 9.11; N, 4.33; O, 9.89. Found: C, 55.62; H, 4.41; N, 4.56. μ_{eff}=4.4 (0.1). X-ray crystals were obtained by layering the solution of ligand L1 in MeCN to the solution of CoCl₂(THF)_{1.5} and LiCl in acetonitrile without stirring at rt in the glove.

2b Ni(L1)Cl₃: Yield: (116 mg, 90%). A dark red solid. M.P.: 292-294° C. (Decomposition). Elementary Analysis (0.974 mg) requires: C, 55.73; H, 4.52; Cl, 16.45; N, 4.33; Ni, 9.08; O, 9.90. Found: C, 55.84; H, 4.52; N, 4.50. μ_{eff}=3.3 (0.1). X-ray crystals were obtained by layering THE into solutions of 2b in DMF.

3a Co(L2)Cl₃: Yield: (87 mg, 69%). A dark red solid. M.P.: 298-300° C. Elementary Analysis (1.080 mg) requires: C, 55.04; H, 4.30; Cl, 16.81; Co, 9.31; N, 4.43; O, 10.11. Found: C, 55.24; H, 4.31; N, 4.59. μ_{eff}=4.3 (0.1). 4 mL MeCN was used for the reaction, only washed with THE and Et₂O. X-ray crystals were obtained by layering the solution of ligand L1 in MeCN to the solution of CoCl₂(THF)_{1.5} and LiCl in acetonitrile without stirring at rt in the glove box.

3b Ni(L2)Cl₃: Yield: (89 mg, 70%). A dark red solid. M.P.: 222-224° C. ¹H NMR (500 MHz, Acetonitrile-d₃, 40° C.) δ 14.90 (br, 1H, Py), 10.26 (br, 1H, Py), 9.39 (br, 1H, Py), 8.93 (br, 1H, Py), 8.00 (dd, J=8.5, 9.5 Hz, 2H, ArH), 7.67 (d, J=9.5 Hz, 2H, ArH), 7.47 (dd, J=8.5, 8.5 Hz, 1H, ArH), 7.07 (d, J=8.5 Hz, 2H, ArH), 6.82 (d, J=8.5 Hz, 2H, ArH), 5.05 (br, 2H, CH₂), 3.60 (s, 6H, OMe), 3.56 (s, 6H, OMe). Elementary Analysis (0.905 mg) requires: C, 55.06; H, 4.30; Cl, 16.81; N, 4.43; Ni, 9.28; O, 10.12. Found: C, 55.23; H, 4.59; N, 4.32. μ_{eff}=2.9 (0.1). 4 mL MeCN was used for the reaction, only washed with THE and Et₂O. X-ray crystals were obtained by layering THE into solutions of 3b in MeCN.

4a Co(L3)Cl₃: Yield: (118 mg, 92%). A dark green solid. M.P.: 218-220° C. Elementary Analysis (1.011 mg) requires: C, 58.01; H, 4.71; Cl, 16.57; Co, 9.18; N, 6.55; O, 4.99. Found: C, 57.98; H, 4.88; N, 8.18. μ_{eff}=4.3 (0.1). X-ray crystals were obtained by layering the solution of ligand L1 in MeCN to the solution of CoCl₂(THF)_{1.5} and LiCl in acetonitrile without stirring at rt in the glove box.

4b Ni(L3)Cl₃: Yield: (99 mg, 77%). A dark green solid. M.P.: 216-218° C. Elementary Analysis (0.847 mg) requires: C, 58.03; H, 4.71; Cl, 16.57; N, 6.55; Ni, 9.15; O, 4.99. Found: C, 58.13; H, 4.82; N, 8.18. μ_{eff}=3.3 (0.1).

Crystallography

All data were collected on an Agilent supernova dual source diffractometer equipped with an Atlas detector, by using CuKa radiation. Data reduction was carried out in the CrysAlis Pro software. Structure solution was made by using direct methods (sir2004), dual-space methods (SHELXT), or charge-flipping (OLEX2). Refinements were carried out in SHELXL within the OLEX2 software.

Cyclic Voltammetry

Voltammetric experiments were performed with a Biologic SP 200 potentiostat connected to a traditional three-electrode cell, which is consisted of an Ag/Ag⁺ reference electrode, a platinum wire counter electrode, and a glassy carbon working electrode. Prior to measurements, the glassy carbon working electrode was polished with alumina slurry (0.05 um), rinsed thoroughly with NERL reagent grade water (Thermo Scientific) between each polishing step, and sonicated in water, followed by a final rinse with acetone, and dried with air or a stream of N₂. All solvents were collected from solvent purification system and solutions were degassed for 5 min before measurements. The cyclic voltammograms curves of L1-L3 (3 mM) and 2-4 (1 mM) in DCM ([TBA][PF₆] 0.1 M) solutions are recorded at a glassy carbon working electrode (n=0.02 V/s).

DFT Calculations

Density functional theory (DFT) calculations were done using the unrestricted hybrid 1993 Becke three-parameter hybrid functional 4-6 with the non-local correlation Lee-Yang-Par (B3LYP).7 The triple-ζ quality basis set LANL2TZ and its correspondent effective core potential was employed for the first row transition metal atoms. The following Gaussian09 internal basis sets were used for non-metal atoms: 6-31G for H, 6-31G(d′) for C, O, and N, and 6-31G(d′,p′) for Cl. The crystal structure of the available complexes were used as starting point for the geometry optimization calculations. The initial structures for complexes XYZ were built using the Spartan modeling software where the C—C bonds in the pyridine alkyl chain were rotated to obtain the desired structure, retaining all the crystallographic bond metrics. Frequency calculations were done to confirm the absence of imaginary frequencies in the geometry optimized structures. The polarizable continuum model (PCM) was employed using the SCRF=PCM keyword and ace-tonitrile as solvent at 298 K. Grimme's empirical dispersion with D3 damping functions were employed for all calculations. Molecular orbitals (MOs) and SCF spin density surfaces were obtained using the standard cubegen utility implemented in Gaussian. The resultant structures and Gaussian cube files were visualized using the Chimera modeling software. All calculations were executed on the Ocelote supercomputer cluster located at the University of Ari-zona High Performance Computing (HPC) center.

Results and Discussion

Carbocation acridinium-based pyridine-containing ligands L1-L2 were synthesized by reacting tris(2,6-dimethoxyphenyl)carbenium tetrafluoroborate 1 with the desired primary amine at room temperature for 2 hours (Scheme B-a). L1 was isolated in 90% yield by reacting 1 and 2-(2-aminoethyl)pyridine in ethyl acetate, and L2 was obtained in 76% yield from the reaction between 1 and 2-(2-aminomethyl)pyridine in acetonitrile.

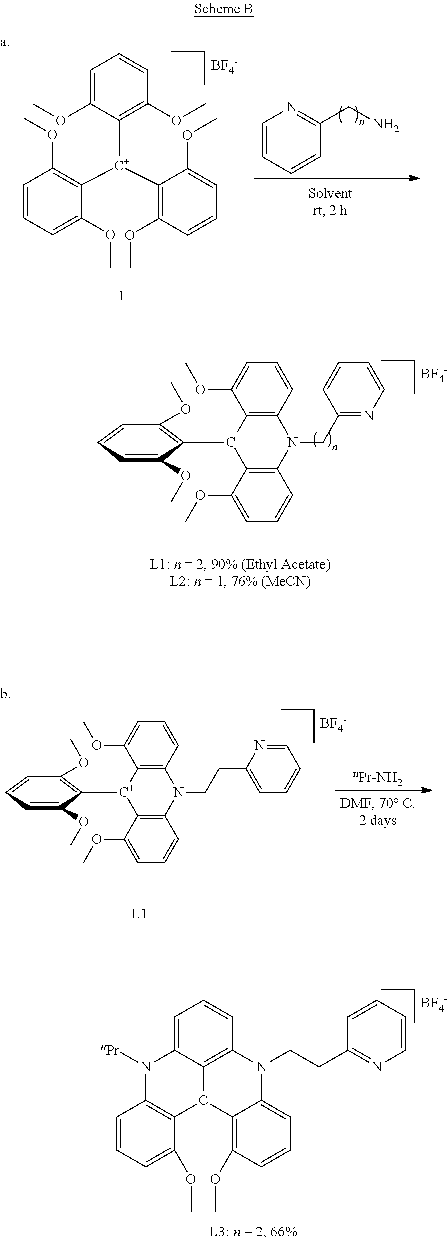

The helicenium L3 was isolated in 66% yield by reacting L1 with an excess of n-propylamine in dimethylformamide (DMF) at 70° C. for 2 days (Scheme B-b). Single crystals of ligand L1-L3 were obtained by layering hexane or Et₂O into solutions of ligands in dichloromethane confirming the formation of the desired product. The pyridine rings in all the three ligands are stretched away from the carbenium scaffolds. No interaction between the pyridine and the carbocation center was observed. However, for L1 and L2, the 2,6-dimethoxyphenyl substituents distort to a small degree against the acridine plan suggesting a weak interaction between the methoxy group and the carbenium center.

The electrochemical behavior of ligands L1-L3 have been studied by cyclic voltammetry (CV) experiments, which were performed using degassed anhydrous DCM solutions with tetrabutylammonium hexafluorophosphate, [TBA][PF₆], as the supporting electrolyte. The reversibility and quasi-reversibility properties of the electrochemical event are supported by the analysis of current density with the square root of the scan rate.

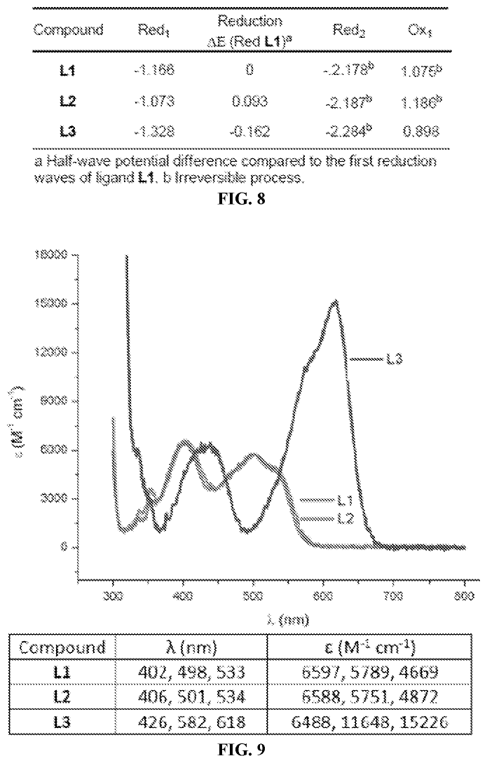

The voltammograms of L1-L3, as well as their electrochemical data versus Fc/Fc⁺ are shown in FIGS. 7 and 8, respectively. In the acridinium cations, an irreversible oxidation of C⁺ to C⁺⁺ occurred at 1.08 V for L1 (1.19 V for L2), followed by a characteristic reversible reduction of C⁺ to C. at E^{red}_{1/2}=−1.17 V (−1.07 V for L2), along with a second irreversible reduction to form C⁻ at −2.18 V (−2.19 V for L2). The difference in redox potential reveals that the acridiunium scaffold in L2 is slightly more electron deficient that L1. This observation is consistent with the electron withdrawing effect of the pyridine group being alpha (L2) or beta (L1) to the heterocyclic ring. The helicenium scaffold L3 displays the same three electrochemical events but at lower potential. Two reductions at E^{red}_{1/2}=−1.33 V (reversible C⁺ to C.) and −2.28 V (irreversible C to form C⁻), and one oxidation at 0.90 V (quasi-reversible C⁺ to C.⁺⁺). Some small events are observed for L1-L3, which were assigned from products generated from C.⁺⁺ or C⁻. The large difference between the E^{red}_{1/2} of ligand L3 compared to L1 further support that acridinium cations are significantly more electron-poor than their helicenium counterpart.

The electronic absorption spectra of L1-L3 were recorded in acetonitrile at r.t. The overlapped spectra as well as a summary of the data are presented in FIG. 5. As can be seen, L1 and L2 have a main absorption band around 400 nm assigned to the local π-π* transition of this scaffold. L1 and L2 also exhibit two absorptions around 500 and 530 nm which consist on an electron transfer from the orthogonal methoxy-substituted aromatic rings to the conjugated cationic systems. Ligand L3 also shows three absorption bands, at 426, 582 and 618 nm, assigned to both localized and delocalized transitions. The two low-energy bands, related to the entire conjugated system, undergo a 120 nm bathochromic shift compared to L1. Without being bound by any theory, it is believed that this indicates a smaller HOMO-LUMO gap consistent with the more electron-rich conjugated helicenium system. These redshifted bands also have dramatical increase of extinction coefficient from 5789 and 4669 M⁻¹ cm⁻¹ in L1 to 11648 and 15226 M⁻¹ cm⁻¹ in L3. The increased of extinction coefficient can be due to the enlargement and twisting of the chromophoric system.

Spectroscopic and electrochemical data show similarities in ligands L1 and L2. Though similar features can be observed in L3, higher electronic density and a decrease in Lewis acid character can be attributed to their lower-energy absorption bands and redox events. Moreover, the intrinsic rigidity in L2 compared to L1 and L3 (methyl-vs ethyl-pyridine), along with the different electronic properties offered by the acridinium and helocenium scaffolds, result in coordination complexes with different coordination modes to the carbenium (Lewis acid) center.

Coordination to Ni and Co

The coordination chemistry of L1-L3 with simple first-row transition-metal halides MCl₂ (M=Co, Ni) was studied. Reacting equimolar amount of ligand L1 and CoCl₂.(THF)_{1.5} in acetonitrile at r.t. under N₂ resulted in a mixture of two paramagnetic species, a dark red compound poorly soluble in acetonitrile and an orange solid soluble in THF. The orange solid was identified as Co(MeCN)₆][BF₄]₂, suggesting an anionic salt exchange with the ligand scaffold. The limited solubility of the obtained red powder in acetonitrile-d₃ and CDCl₃ was insufficient to confirm the paramagnetic nature due to hampering of proper NMR spectroscopy analysis. However, when dissolved in protic or strongly coordinating solvent, such as MeOH-d₄, D₂O or DMSO-d₆, free ligand L1 was observed via ¹H NMR spectroscopy, with no signal for [BF₄] observed in the ¹⁹F NMR spectrum. These observations suggest the successful formation of a metal complex containing L1 and the Cl/BF₄ anion exchange. In order to optimize the reaction and prevent the formation of side product, 2.0 molar equivalents of LiCl were added to the reaction mixture. Under this condition, the reaction proceeded to completion, affording the desired Co(II) complex (2a) in 84% yield (Scheme C). Following the same procedure, the corresponding Co(II) complexes 3a and 4a were obtained upon coordination of L2 and L3, respectively. Similarly, Ni(II) complexes 2b, 3b and 4b were synthesized by reacting NiCl₂(glyme) with the corresponding ligands L1-L3. Complexes 2-4 have poor solubility in acetonitrile and crashed out from the solution. The resulting powders were filtered and washed with MeCN, THE and Et₂O to remove residual LiCl, LiBF₄ and unreacted starting material. Due to a relatively higher solubility in MeCN, 3a,b were only washed with THE and Et₂O. Isolation of the microcrystalline powder afforded the desired complexes 2-4 in moderate to good yields (Scheme 3). Sample purities have been confirmed by elemental analysis. Magnetic susceptibility measurements in the solid state at r.t. are consistent with: i) Co(II) d⁷ and Ni(II) d⁸ metallates in a tetrahedral environment and a positively charged carbocation ligand, or ii) Co(III) d⁶ and Ni(III) d⁷ in a tetrahedral environment and a neutral carboradical ligand with antiferromagnetic coupling between the metal and the unpair electron centered on the ligand.

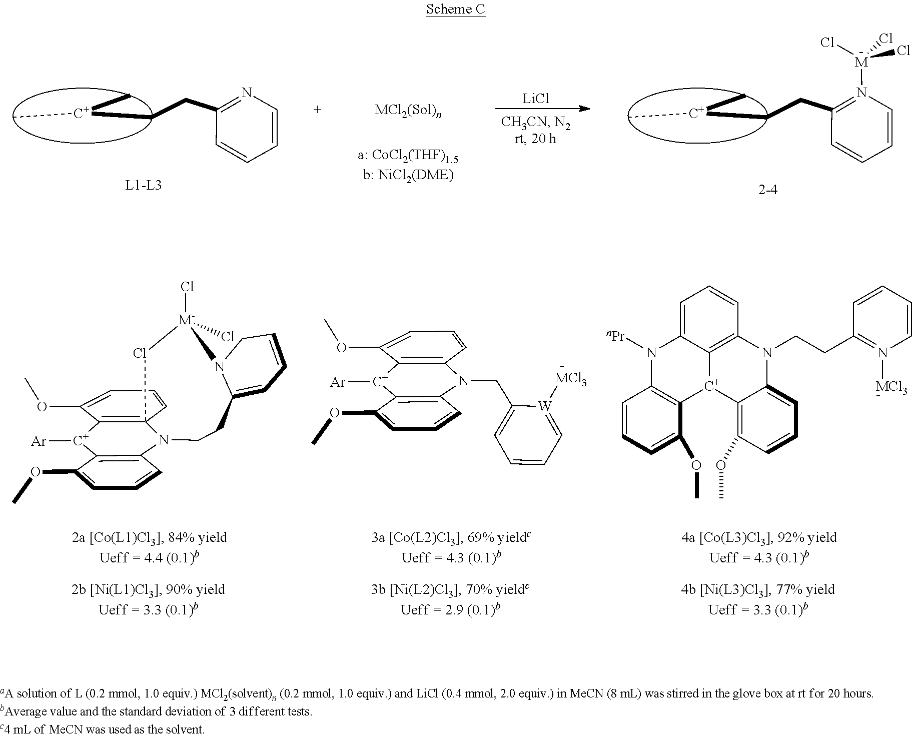

Due to a relatively better solubility in CD₃CN, 3b was analyzed by ¹H NMR spectroscopy. Compared to the free ligand L2, the protons near the paramagnetic Ni center exhibits broader signals and small but significant chemical shifts changes. The four pyridinic protons are shifted from 7.4-8.3 ppm to 8.9-14.9 ppm, and the methylene protons from 6.70 ppm to 5.05 ppm. However, the protons from the heterocyclic fused aromatic system were not significantly affected, suggesting that spin density is localized at the metal center.

Solid state structural analysis. X-ray quality single crystals of cobalt(II) complexes 2a-4a were obtained by slow diffusion of a solution of MeCN containing L1-L3 into a mixture of CoCl₂(THF)_{1.5} and LiCl in acetonitrile. Nickel complexes 2b and 3b were crystallized by diffusion of a THE layer into a solution of the complexes in DMF.

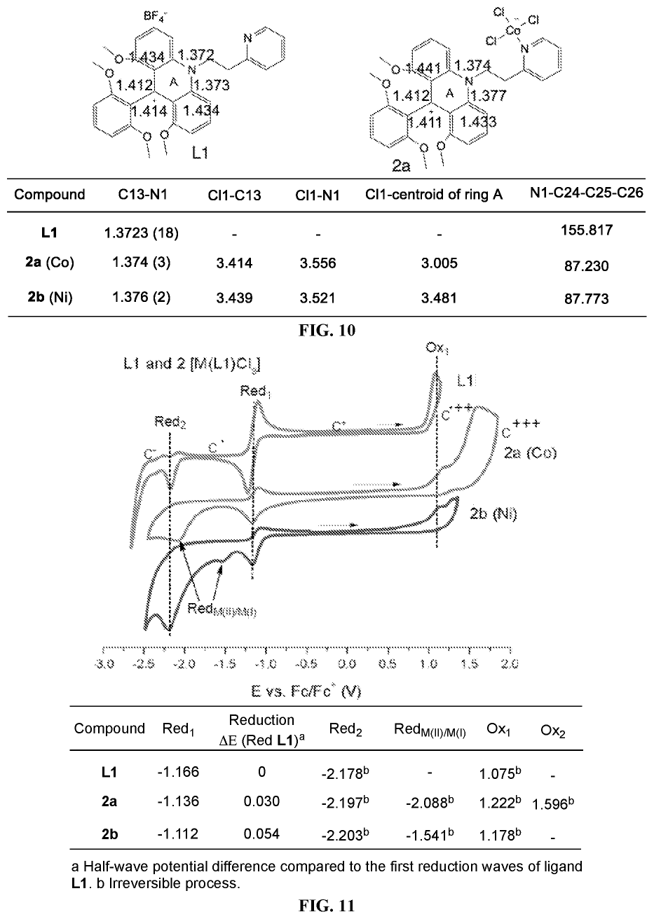

X-ray diffraction analysis confirmed the formation of zwitterion metallate trichloride complexes bound to the cationic ligand via the pyridine anchor in which the metal center adopts a canonical tetrahedral geometry. Due to the difference in rigidity and Lewis acidity between the ligands L1-L3, different modes of coordination are observed in the solid state. The flexible and relatively electron poor carbenium L1 favored a coordination mode of complexes 2 in which one of the chloride resides above the heteroaromatic carbenium ring (ring A) (FIG. 10). Bond distances between the centroid of this ring and Cl(1) are 3.005 Å and 3.481 Å for complexes 2a and 2b, respectively. Additionally, large torsion angels can be found for N(1)-C(24)-C(25)-C(26) in metal complexes 2. However, despite the close proximity of Cl(1) with C(13) (3.414 Å and 3.439 for 2a and 2b respectively) compare to the rest of the elements of the ring A, no significant elongation of the bond lengths between C(13) and the rest of the scaffold was observed between L1 and 2. It is believed that this coordination environment suggests the presence of a Cl—C⁺ interaction strong enough to overcome the geometrical strain imposed by the folding of the metallate scaffold above the heterocyclic carbenium rings. However, this interaction between the metallate core and the positively charged scaffold is best described as an ionic Van-der-Walls interaction. Complex 2 can be best described with the coordination Mode II—Lewis acid coordinates with a co-ligand.

On the other hand, with a shorter linker (L2) or more electron rich scaffold (L3), no intramolecular interaction is observed and the metallate core sits away from the carbenium scaffold. In complexes 3 and 4 no distorsion or changes of bond lengths are observed compare to L2 and L3, respectively. Complexes 3-4 are therefore best described with the coordination Mode I in FIG. 1—Lewis acid remains as a pendant part, in which no interaction between the carbocation with the metallate core. Finally, complexes 2-4 have similar structural metrics around the metal centers compare to each other and to reported LMCl₃ (L=Pyridine or amine) further supporting that the interaction in 2 is weak and non-covalent (means values of Co—Cl=2.26, Ni—Cl=2.24, Co—N=2.09 and Ni—N=2.04 Å).

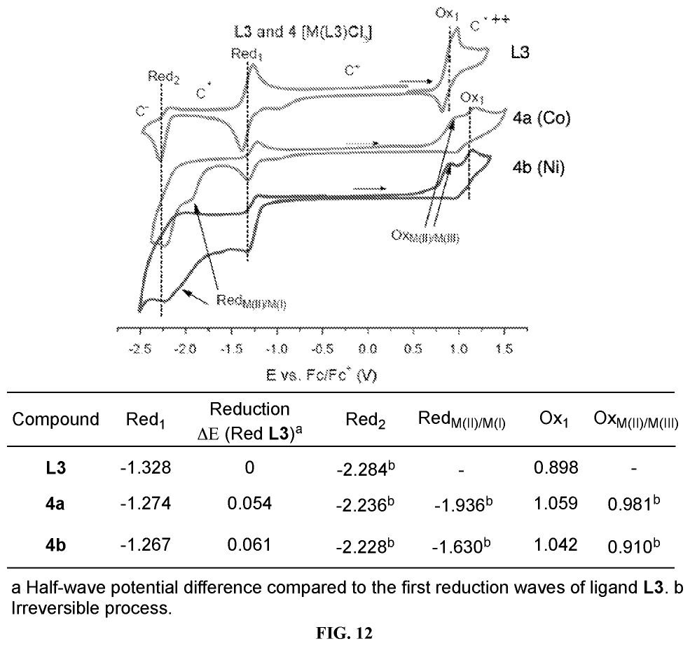

Electrochemical properties. The electrochemical behavior of the metal complexes 2-4 have been studied by cyclic voltammetry (FIGS. 11 and 12). Complexes 2a and 2b featured similar redox events with L1: a reversible reduction around −1.1 V, an irreversible reduction around −2.2 V, along with an irreversible oxidation around 1.2 V, which are assigned as ligand-based processes. A second fully irreversible oxidation is observed for 2a at 1.60 V, which is believed to be result from generation of C⁺⁺⁺ from C.⁺⁺. Due to a smaller window for ligand L1 and complex 2b, no such event is observed, but similar process occurs at 1.61 V for complex 3b as well. In addition, a new irreversible reduction is observed for complexes 2a at −2.09 V and 2b at −1.54 V, which can be assigned to metal-based reduction from M(II) to M(I). Complexes 3 exhibited a similar behavior as 2. Similarly, for complexes 4 all the ligand-based redox events were observed, as well as, the irreversible reduction event of M(II) to M(I) at −1.94 V (4a) and −1.63 V (4b). However, an additional new irreversible oxidation was observed at 0.98 V for the cobalt complex 4a and 0.91 V for the nickel complex 4b, which may be assigned as the metal base oxidation of M(II) to M(III).

Overall, these metal complexes show the three characteristic ligand-based redox events: i) irreversible (L1,L2) or quasi-reversible (L3) oxidation of C⁺ to C.⁺⁺, ii) reversible reduction of C⁺ to C., and iii) an irreversible reduction of C. to C⁻. The reversible reduction of C⁺ to C. and oxidation C+/C+. of 2-4 happen at a more positive potential compared to the free ligands (ΔE˜50 mV for C⁺/C. and ΔE˜150 mV for C⁺/C⁺⁺.). In addition, for cobalt complexes 2a, 3a and 4a, a reduction event of Co(II) to Co(I) is observed between −1.88 to −2.09 V, while the reduction of Ni(II) to Ni(I) occurs between −1.54 to −1.62 V complexes 2b, 3b and 4b. The reduction potentials are more negative compared to reported neutral Co(II)/Co(I) and Ni(II)/Ni(I), consistent with the metallate form of 2-4. The irreversible oxidation event assigned to M(II)/M(III) couple is only observed with the electron rich helicenium complexes 4a and 4b.

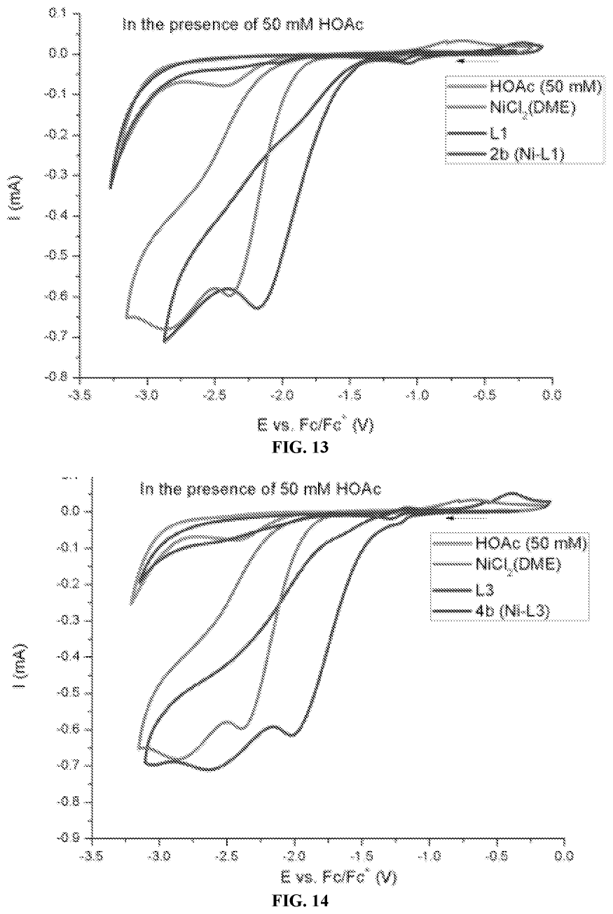

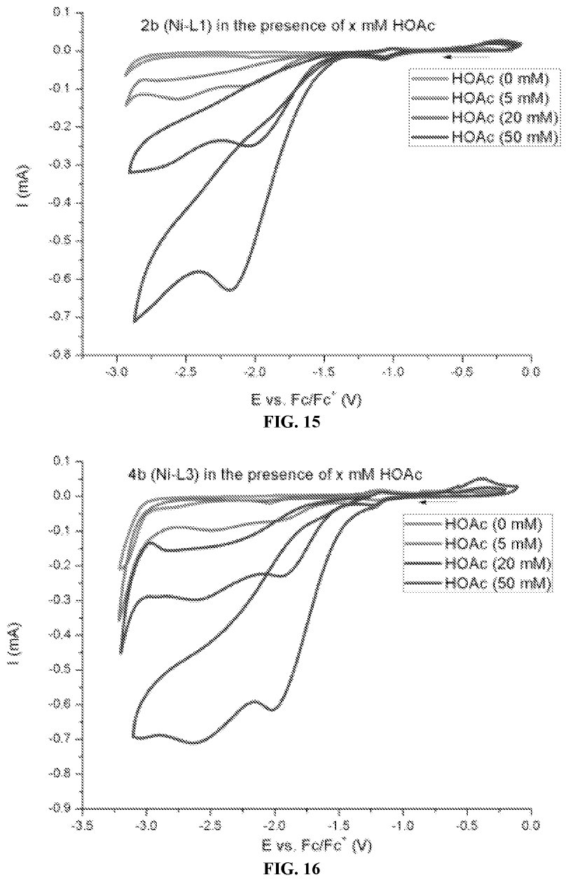

Electrocatalytic studies with acetic acid. The reduction potential of the Ni(II)/Ni(I) couple at around −1.5 V offer the opportunity to evaluate the electrocatalytic properties of 2b-4b toward proton reduction. Cyclic voltammetry of 2b and 4b in presence of various amount of acidic acid showed a catalytic current response. FIGS. 15 and 16, respectively. The bare glassy carbon electrode, NiCl₂(DME), complexes 2b and 4b and ligands alone were run under the similar conditions and compared. FIGS. 13 and 14, respectively. All three complexes as well as NiCl₂(DME) exhibited catalytic activity. A significant shift of the overpotential was observed for some compounds (e.g., up to 370 mV for 4b). The catalytic peak reduction of acetic acid occurred at −2.35 V for NiCl₂(DME) and at around −2.18, −2.24 and −2.02 V, for 2b, 3b and 4b respectively. The lower overpotential observed in 2b-4b compared to NiCl₂(DME) suggest that the ambiphilic ligand scaffold facilitate electron transfer from the metal center.

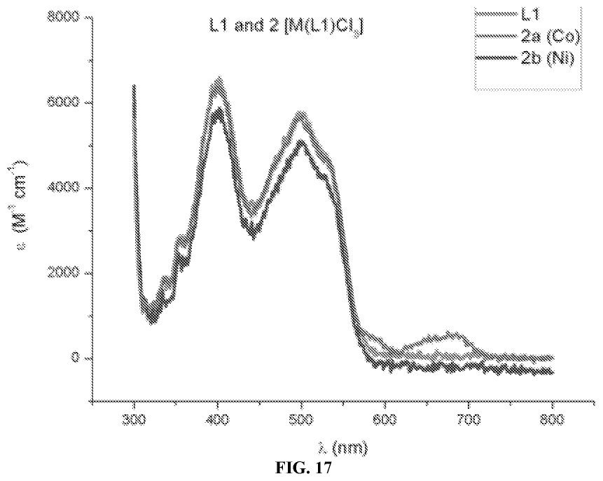

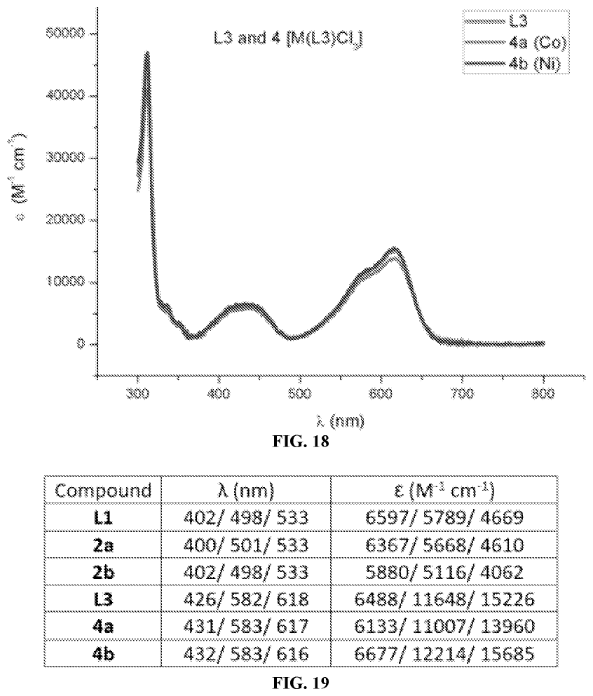

Spectroscopic properties. Electronic absorption spectra of the metal complexes 2 and 4 (con. 10-5 M) were recorded in acetonitrile at RT. The overlapped spectra with the corresponding ligands L1-L3 as well as a summary of the data are shown in FIGS. 17-19. The cobalt and Ni complexes 2 have similar absorption properties with the free ligand L1, with three main absorptions at 400, 501 and 533 nm for 2a and 402, 498 and 533 nm for 2b, albeit slightly lower extinction coefficients (FIG. 19). In addition, broad peaks with low extinction coefficient were observed between 600-700 nm for both Co(II) complex 2a and 3a (FIG. 17). These Laporte relaxed transitions are characteristic e to t₂ transition for tetrahedral cobalt trichloride species and are in agreement with analog complex such as the Co(pyridine)Cl₃⁻, which supports the formation of zwitterion metallate trichloride M(II) bound to cationic ligand. Complexes 4 show very slight changes on the spectrum compared to ligand L3, regarding to either main absorption peaks or extinction coefficients. As can be seen, the Co and Ni complexes 2-4 display similar absorption properties to the corresponding carbenium free ligand. Without being bound by any theory, this suggests: i) no electron transfer from the metal center to the heterocyclic system occurs and ii) the ionic Van-der-Walls interactions observed in the solid state is not present in the solution at RT.

DTF calculations. Density functional theory (DFT) calculations were performed on complexes 2a and 2b to better understand the interaction of the carbenium center with the transition metal halide observed in the solid state (ionic interaction or crystal packing). Solvated and gas-phase models were calculated to obtain a better representation of the intramolecular forces present during the structural characterization process. Isoelectronic models were calculated with the metal-halide fragment in-plane and out-of plane of the carbenium center. First, calculation was performed without empirical dispersion. Under this condition, the out of plane was found to be more thermodynamically stable in both solution and gas phase by 8.6 and 4.7 Kcal mol⁻¹ respectively. When the Grimme's empirical dispersion with D3 damping functions were employed, the solvated model shows a thermoneutral equilibrium between the in and out of plane conformation, with the out of plane being more thermodynamically stable (ΔE=1.02 Kcal mol⁻¹). In the gas-phase, this energy difference increases to 4.81 Kcal mol⁻¹, with the in-plane configuration being favoured. Similar trends in the energy difference between solvated and gas-phase models were observed for the Ni complex 2b, showing no effect on the nature of the first-row transition metal center for a preferred in/out of plane structural configurations.

The thermoneutral equilibrium in solution and the slightly favoured in-plane configuration in the gas phase are both in agreement the experimental observations: i) the lack of interaction observed in solution by UV spectroscopy, ii) the crystallization of the in-plane configuration observed by X-ray diffraction analysis.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A compound comprising a moiety of the formula: 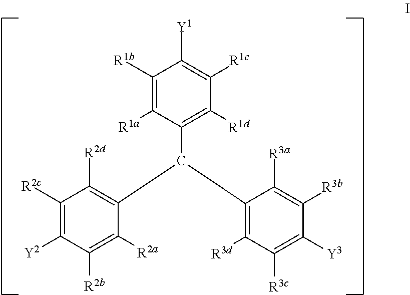
wherein
said moiety of formula I is a radical, a cation, or a radical dication;
each of R^{1a}, R^{1b}, R^{1c}, R^{1d}, R^{2a}, R^{2b}, R^{2c}, R^{2d}, R^{3a}, R^{3b}, R^{3c}, and R^{3d} is independently H, halide, haloalkyl, —NR^{a}R^{b}, C₁-C₁₂ alkyl, C₁-C₄ alkoxy, —NO₂, —CN, —CO₂R (wherein R is H or C₁-C₄ alkyl), or Ar¹;
or R^{2a} and R^{3d} together form —X¹—;
or R^{1a} and R^{2d} together form —X²—;
or R^{1d} and R^{3a} together form —X³—;
each of R^{a} and R^{b} is independently H or C₁-C₄ alkyl;
each of X¹, X² and X³ is independently O, NR^{4a}, PR^{4a}, CR^{4a}R^{4b}, or SiR^{4a}R^{4b};
or R^{1a} and R^{1b} together with atoms to which they are attached form an optionally substituted aromatic ring;
or R^{2c} and R^{2d} together with atoms to which they are attached form an optionally substituted aromatic ring;
each of Y¹, Y², and Y³ is independently H, OR^{5a}, CR^{5a}R^{5b}R^{5b}, NR^{5a}R^{5b} PR^{5a}R^{5b} NO₂, CN, haloalkyl, CO₂R, N3, or Ar¹;
each of R^{4a}, R^{4b}, R^{5a}, and R^{5b} is independently H, halide, haloalkyl, C₁-C₁₂ alkyl, C₁-C₄ alkoxy, —NR^{a}R^{b} where at least one of R^{a} and R^{b} is C₁-C₄ alkyl, Ar¹, or a moiety of the formula -L-Z,
wherein
L is a linker; and
Z is a heterocyclic species of Formula I, a coordinating group able to bind or complex to a metal ion, or a water-soluble group; and
Ar¹ is optionally substituted aryl having from 0 to 5 substituents, each of which is independently selected from the group consisting of X¹, haloalkyl, NR^{a}R^{b}, C₁-C₄ alkyl, and C₁-C₄ alkoxy, heteroaryl, fused aryl or Ar¹,
provided when said Formula I is a cation then
(i) when R^{2a} and R^{3d} together form —X¹—; R^{1a} and R^{2d} together form —X²—; and R^{1d} and R^{3a} together form —X³—, then no more than one of X¹, X², and X³ is O;
(ii) when R^{2a} and R^{3d} together form —X¹—, and R^{1a} and R^{2d} together does not form —X²—; and R^{1d} and R^{3a} together does not form —X³—, then at least one of R^{1a}, R^{1b}, R^{1c}, R^{1d}, or Y¹ is not H, halogen, or C₁-C₁₂ alkyl;
(iii) at least one of (a) R^{2a} and R^{3d} together form —X¹—; (b) R^{1a} and R^{2d} together form —X²—; or (c) R^{1d} and R^{3a} together form —X³—; and
(iv) when R^{2a} and R^{3d} together form —X¹—; R^{1a} and R^{2d} together form —X²—; and R^{1d} and R^{3a} together form —X³—, then none of X¹, X², and X³ is NR^{4a}.

2. The compound of claim 1, wherein
Y¹ is NR^{5a}R^{5b}, PR^{5a}R^{5b}, haloalkyl, N₃, or Ar¹; or
L is C₁-C₁₀ alkylene, alkynylene, alkenylene, arylene, or heteroarylene; or
each of R^{1c}, R^{2c}, and R^{3c} is independently C₁-C₄ alkoxy; or
Y¹, R^{1a}, R^{1b}, R^{2a}, R^{2b}, R^{3a}, and R^{3b} are H.

3. The compound of claim 1, wherein said moiety of Formula I is selected from the group consisting of:

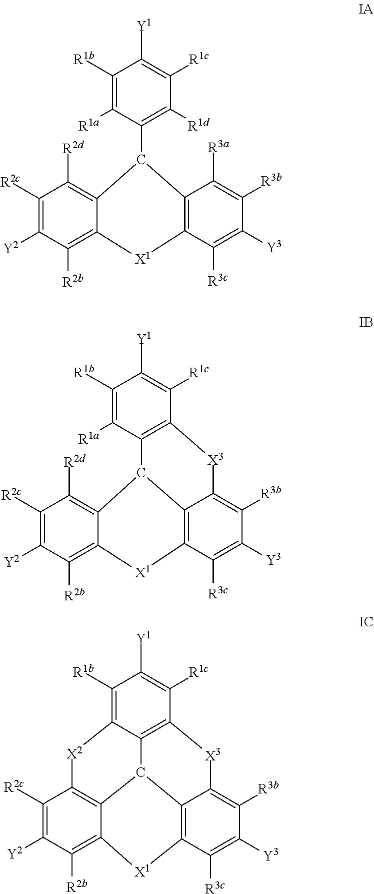

4. The compound of claim 3, wherein said moiety of Formula IB or IC is a radical or a radical dication.

5. The compound of claim 3, wherein X¹ is NR^{4a}.

6. The compound of claim 5, wherein R^{4a} is said moiety of the formula L-Z.

7. The compound of claim 1, wherein Z is a coordinating group selected from the group consisting of bipyridinyl, pyridinyl, —PR₂, —OPR₂, —NHC, —NR₂, diimine, imine, —OH, —OR, —SR, —SH, diphosphines, —RNC, —CO₂H, and carboiimine, and wherein each R is independently C₁-C₁₂ alkyl or Aryl.

8-10. (canceled)

11. A compound comprising a radical or a diradical cation moiety of the formula: 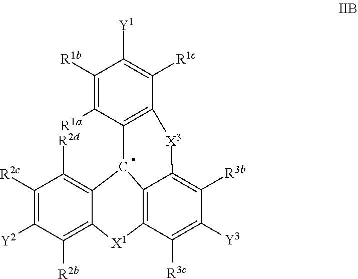
wherein
each of R^{1a}, R^{1b}, R^{1c}, R^{2b}, R^{2c}, R^{2d}, R^{3b}, and R^{3c} is independently H, halide, haloalkyl, —NR^{a}R^{b}, C₁-C₁₂ alkyl, C₁-C₄ alkoxy, —NO₂, —CN, —CO₂R (wherein R is H or C₁-C₄ alkyl), or Ar¹;
or R^{1a} and R^{2d} together form —X²—;
each of R^{a} and R^{b} is independently H or C₁-C₄ alkyl;
each of X¹, X² and X³ is independently O, NR^{4a}, PR^{4a}, CR^{4a}R^{4b}, or SiR^{4a}R^{4b};
or R^{1a} and R^{1b} together with atoms to which they are attached to form an optionally substituted aromatic ring;
or R^{2c} and R^{2d} together with atoms to which they are attached to form an optionally substituted aromatic ring;
each of Y¹, Y², and Y³ is independently H, OR^{5a}, NR^{5a}R^{5b}, PR^{5a}R^{5b}, NO₂, CN, CF₃, CO₂R, N₃, or Ar¹;
each of R^{4a}, R^{4b}, R^{5a}, and R^{5b} is independently H, halide, haloalkyl, C₁-C₁₂ alkyl, C₁-C₄ alkoxy, —NR^{a}R^{b} where at least one of R^{a} and R^{b} is C₁-C₄ alkyl and R^{4a} is not attached to N, Ar¹, or a moiety of the formula -L-Z,
wherein
L is a linker; and
Z is a coordinating group able to bind or complex to a metal ion; and
Ar¹ is optionally substituted phenyl having from 0 to 5 substituents, each of which is independently selected from the group consisting of X¹, haloalkyl, NR^{a}R^{b}, C₁-C₄ alkyl, and C₁-C₄ alkoxy.

12. (canceled)

13. The radical moiety of claim 11, wherein each R^{4a} is independently C₁-C₁₂ alkyl or a moiety of the formula -L-Z.

14. The radical moiety of claim 13, wherein Z is selected from the group consisting of a heteroaryl, heterocyclyl, and a heteroatom functional group.

15. The radical moiety of claim 11 wherein
Y¹, Y², and Y³ are NR^{5a}R^{5b}, wherein each of R^{5a} and R^{5b} is independently H, haloalkyl, or C₁-C₁₂ alkyl, provided at least one of R^{5a} and R^{5b} is not H, or
R^{1a} and R^{2d} are C₁-C₄ alkoxy; or
X¹ and X³ are NR^{4a}.

16. (canceled)

17. A photocatalytic compound capable of catalyzing an oxidative reaction and/or a reductive reaction, said photocatalytic compound comprising a compound of claim 1.

18-30. (canceled)

31. A compound comprising a carbocation of the formula: 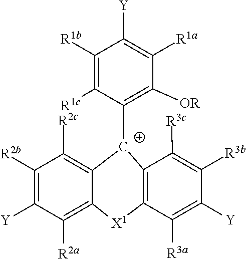
wherein
R is C₁-C₁₂ alkyl or Aryl;
X¹ is O, NR^{4a} PR^{4a}, CR^{4a}R^{4b}, or SiR^{4a}R^{4b};
each of Y is independently H, OR^{5a}, NR^{5a}R^{5b} PR^{5a}R^{5b} NO₂, CN, haloalkyl, N₃ CO₂R, or Ar¹;
each of R^{1a}, R^{1b}, R^{1c}, R^{2a}, R^{2b}, R^{2c}, R^{3a}, R^{3b}, and R^{3c}, is independently H, halide, CF₃, NH₂, C₁-C₁₂ alkyl, C₁-C₄ alkoxy, C₁-C₄ alkylamino, C₁-C₄ dialkyl amino, NO₂, CN, CO₂R, or Ar¹;
each of R^{4a}, R^{4b}, R^{5a}, and R^{5b} is independently H, halide, CF₃, C₁-C₁₂ alkyl, C₁-C₄ alkoxy, C₁-C₄ alkylamino, C₁-C₄ dialkyl amino, or Ar¹;
or R^{1c} and R^{2c} together form —X²—;
or OR and R^{3c} together form —X³—;
each of X² and X³ is independently O, NR^{4a}, PR^{4a}, CR^{4a}R^{4b}, or SiR^{4a}R^{4b};
or R^{1b} and R^{1c} together with atoms to which they are attached form an optionally substituted phenyl;
or R^{2b} and R^{2c} together with atoms to which they are attached form an optionally substituted phenyl; and
Ar¹ is optionally substituted phenyl having from 0 to 5 substituents, each of which is independently selected from the group consisting of X¹, CF₃, NH₂, C₁-C₄ alkyl, C₁-C₄ alkoxy, C₁-C₄ alkylamino, and C₁-C₄ dialkyl amino; or
at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, wherein L is a linker; and Z is a coordinating group that is capable of coordinating to a metal complex.

32. The compound of claim 31, wherein at least one of R^{1a}, R^{1b}, R^{1c}, R^{2a}, R^{2b}, R^{2c}, R^{3a}, R^{3b}, R^{3c}, R^{4a}, R^{4b}, R^{5a}, and R^{5b} is a moiety of the formula -L-Z, wherein L is a linker; and Z is a coordinating group that is capable of coordinating to a metal complex.

33. The compound of claim 31, wherein
said compound is of the formula: 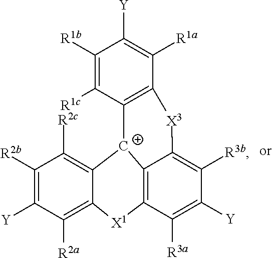
or
said compound is of the formula:

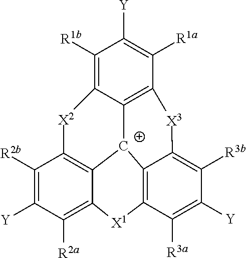

34. (canceled)

35. The compound of claim 31, wherein X¹ is NR^{4a}.

36. The compound of claim 34, wherein R^{4a} is said moiety of the formula L-Z.

37. The compound of claim 31, wherein Z is a coordinating group selected from the group consisting of bipyridinyl, pyridinyl, —PR₂, —OPR₂, —NHC, —NR₂, diimine, imine, —OH, —OR, —SR, —SH, diphosphines, —RNC, —CO₂H, and carboiimine, and wherein each R is independently C₁-C₁₂ alkyl or Aryl.

38. The compound of claim 37, wherein Z is coordinated or complexed to a metal complex.

39-41. (canceled)

42. The compound of claim 31, wherein
L is C₁-C₁₀ alkylene, alkynylene, alkenylene, arylene, or heteroarylene; or
each of R^{1c}, R^{2c}, and R^{3c} is independently C₁-C₄ alkoxy; or
Y, R^{1a}, R^{1b}, R^{2a}, R^{2b}, R^{3a}, and R^{3b} are H.

43-57. (canceled)
